(19) United States
(12) Patent Application Publication
(10) Pub. No.: US 2017/0224199 A1
(43) Pub. Date: Aug. 10, 2017

(54) METHOD AND APPARATUS FOR STEERABLE, ROTATABLE, MICROENDOSCOPE WITH TOOL FOR CUTTING, COAGULATING, DESICCATING AND FULGURATING TISSUE

(71) Applicant: Research & Development International, Inc., Pasadena, CA (US)

(72) Inventors: Joseph R. Demers, Pasadena, CA (US); Marek Sekowski, Pacific Palisades, CA (US)

(21) Appl. No.: 15/493,916

(22) Filed: Apr. 21, 2017

Related U.S. Application Data
(62) Division of application No. 14/210,126, filed on Mar. 13, 2014, now abandoned.
(60) Provisional application No. 61/786,490, filed on Mar. 15, 2013.

(51) Int. Cl. A61B 1/00; A61B 17/32; A61B 1/07; A61B 1/018; A61B 1/005

(52) U.S. Cl. CPC A61B 1/00147; A61B 1/00165; A61B 1/00066; A61B 1/018; A61B 1/00133; A61B 1/0057; A61B 1/0052; A61B 1/00096; A61B 1/07; A61B 17/320016

(57) ABSTRACT

An exemplary embodiment providing one or more improvements includes a micro endoscope having steering, rotation and tool control function which can be utilized for insertion using a needle and catheter for performing arthroscopy and endoscopic procedures.

### RELATED APPLICATIONS

The present application is a divisional of U.S. patent application Ser. No. 14/210,126, filed on Mar. 13, 2014 which itself claims priority from U.S. Provisional Patent Application Ser. No. 61/786,490, filed on Mar. 15, 2013, each of which applications are hereby incorporated by reference in their entireties.

### BACKGROUND

Endoscopes have continued to evolve since their inception in the 1800's because of their utility and versatility. Medical endoscopes can be used for performing medical procedures which can include viewing and manipulating tissues in body cavities. While relatively large endoscope probes can be used in existing body channels for some types of procedures, small endoscope probes (FIG. 1) can be used to perform intricate surgery through small incisions. Termed micro-invasive because of the small incisions, patient recovery time and surgical complications are significantly reduced when compared to similar procedures using non-endoscopic techniques. More recently, endoscopes with diameters of less than 1 mm have made it possible to gain access to the body cavity through a large gauge needle or catheter as opposed to an incision. In some cases, as with mammary duct examination and biopsy, penetrating the skin is not necessary with an endoscope small enough to enter the dilated mammary duct.

In general terms, an endoscope employs a flexible bundle of glass fibers (FIG. 2a) to transmit an image from the distal end to the proximal end. This bundle of fibers is typically referred to as an imaging fiber and current technology makes it possible to construct a sub-millimeter diameter imaging fiber that incorporates thousands of individual fibers. The individual fibers of the bundle may also be referred to as elements of the imaging fiber bundle, see inset FIG. 2a. The element size and density determines the pixel size for the transmitted image and the flexibility of the imaging fiber bundle. For instance, Fujikura's FIGH-10-350N has an outer diameter of 0.35 mm and is a bundle of ten thousand 3.5 um diameter fibers. During imaging, each of these elements acts as a pixel for the image and transmits this pixel via internal reflection from the distal end to the proximal end (FIG. 2b). The Fujikura bundles are available in many different diameters and element counts, however, the element density remains roughly the same. This is fundamental and due to the nature of transmitting white light along a fiber and minimizing color dispersion. Smaller individual fibers would increase the fiber density, but the fibers would have greater loss at longer wavelengths. They would also be significantly more difficult to manufacture.

The imaging fiber is spatially coherent meaning that there is a one-to-one correspondence between the position of the elements on the input of the bundle and on the output of the bundle (FIG. 2b). This makes it possible to transmit an image along the bundle. If the elements were not spatially coherent, and elements which change their relative positions along the length of the imaging fiber bundle, an image transmitted through the bundle would exit the bundle with the spatial information distorted (i.e. a different image would be formed) (FIG. 3). While the image fiber is spatially coherent with itself, this is not to say that the pattern of elements in the bundle follows a specific pattern. The positions are not defined by a pattern and are fairly random as to where the centers of the individual fibers are positioned.

While the ability to image tissue is valuable, the greater utility of an endoscope is the ability to perform intricate surgical procedures at remote locations in the body. Therefore, a conventional endoscope has at least one working channel that extends from the distal end to a proximal end and may be used to deliver tools to the site being imaged (FIG. 4). Most modern endoscopes, however, have several working channels that are employed for various functions: fluid delivery and removal, forceps and clamps just to name a few. As the endoscope size, and in particular the working channel size, is reduced to sub-millimeter dimensions, the ability to clean the working channel between uses becomes impossible and the endoscope must therefore be disposed of after each use to prevent cross-contamination between patients. Several techniques are available to avoid the expense of throwing away the entire scope have developed including incorporating the working channel into a disposable sheath that slides into place over the more expensive optics. This allows the optics to be reused, but they must still be sterilized to prevent cross-contamination if the sheath should leak.

The foregoing examples of the related art and limitations related therewith are intended to be illustrative and not exclusive. Other limitations of the related art will become apparent to those of skill in the art upon reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In general, a method and associated apparatus are described for an endoscope which includes a probe having an imaging fiber bundle for transferring a light image. The imaging fiber bundle having a distal end for receiving the light image and a proximal end portion extending out of the probe for emitting the light image. The endoscope including a handle arrangement connected to the probe and configured to support part of the proximal end portion of the imaging fiber bundle for twisting therealong responsive to rotating the probe including the distal end of the imaging fiber bundle relative to the handle arrangement.

In another embodiment, an endoscope includes a probe that is configured for insertion into tissue. An imaging fiber bundle is supported by the probe and includes a distal end, a proximal end, and a length therebetween. The imaging fiber bundle is configured for receiving a light image using the distal end, transferring the light image from the distal end to the proximal end, and emitting the light image from the proximal end. The endoscope also including a handle arrangement connected to the probe and the imaging fiber bundle. The handle arrangement is configured for co-rotating the probe and a distal portion of the imaging fiber bundle while holding a proximal portion of the imaging fiber bundle substantially without rotation to rotate the light image along the length responsive to the probe rotation such that the light image as emitted from the proximal end of the imaging fiber bundle is rotated relative to the light image received at the distal end of the imaging fiber bundle.

In another embodiment, an endoscope is disclosed having a probe configured for insertion into tissue and an imaging fiber bundle that is supported by the probe and having a distal end, a proximal end, and a length therebetween. The imaging fiber bundle is configured for receiving a light image using the distal end and for transferring the light image from the distal end to the proximal end, and emitting the light image from the proximal end. A handle arrangement is connected to the probe and the imaging fiber bundle. The handle arrangement is configured for co-rotating the probe and a distal portion of the imaging fiber bundle while holding a proximal portion of the imaging fiber bundle substantially without rotation to rotate the light image along the length responsive to the probe rotation such that the light image as emitted from the proximal end of the imaging fiber bundle is rotated relative to the light image received at the distal end of the imaging fiber bundle.

In yet another embodiment, an endoscope is disclosed which includes a probe having a distal end configured for insertion into tissue and a proximal end configured for use outside of the tissue. The probe defines a working channel for guiding endoscopic tools from the proximal end of the probe to the distal end of the probe. An endoscope tool is configured for insertion through the working channel to a surgical site in the tissue and for tool actuation to manipulate tissue at the surgical site, the tool and the working channel including complementary configurations which cooperate for the tool actuation of the endoscope tool.

In still another embodiment, an endoscope tool is disclosed having an elongated pull cable assembly including a proximal end and a distal end. The pull cable assembly having a flexible inner cable and a cable housing surrounding a portion of a length of the inner cable such that the inner cable is movable lengthwise within the cable housing. A tool head is operatively connected to the distal end of the pull cable assembly for selective actuation by lengthwise movement of the inner cable within the cable housing at the proximal end of the pull cable assembly. An actuator is connected to the proximal end of the pull cable assembly to actuate the tool head by moving the inner cable lengthwise within the cable housing. The actuator including a core arrangement having proximal and distal core sections positioned along a common elongation axis and separated by a break that is defined therebetween. The proximal core section is configured for connection to the proximal end of one of the inner cable and the cable housing, and the distal core section configured for connection to the proximal end of the other one of the inner cable and the cable housing. The actuator includes a shell arrangement connected to the core arrangement and configured for collapsible movement toward the core arrangement in a way that expands the break between the core sections along the elongated axis to move the inner cable lengthwise in the cable housing to operate the tool head.

In another embodiment, an endoscope is disclosed including a tool assembly having a tool head that is configured for selective movement to manipulate tissue and a tool head actuator that is connected to selectively move the tool head using a cable assembly having a cable sheath and an inner cable that moves longitudinally in the cable sheath. An elongated probe is includes a distal end configured for insertion into tissue and a proximal end configured for use outside of the tissue. The probe defines a working channel for guiding the tool head from the proximal end of the probe to the distal end of the probe while the tool head actuator remains outside of the tissue. A handle assembly is connected to the probe. The handle assembly includes a handle body, a trigger arrangement and a latching mechanism. The latching mechanism is configured for selectively connecting the tool assembly to the handle assembly and the trigger arrangement is configured for an actuating movement relative to the handle body to actuate the cable assembly to bend the probe near the distal end of the probe and an unlatching movement relative to the handle body to control the latching mechanism to disconnect the tool assembly from the handle assembly.

In yet another embodiment, an endoscope is disclosed including a tool assembly having a tool head that is configured for selective movement to manipulate tissue and a tool head actuator that is connected to selectively move the tool head using a cable assembly having a cable sheath and an inner cable that moves longitudinally in the cable sheath. An elongated probe including a distal end is configured for insertion into tissue and a proximal end configured for use outside of the tissue. The probe defines a working channel for guiding the tool head from the proximal end of the probe to the distal end of the probe while the tool head actuator remains outside of the tissue and the distal end is configured for selective bending. A handle assembly is operatively coupled to the probe. The handle assembly includes a handle body and a trigger arrangement that is configured for an actuating movement relative to the handle body to actuate the cable assembly to initially extend the tool head from the probe and, thereafter, bend the distal end of the probe.

In yet another embodiment, an endoscope tool is disclosed that includes a set of forceps jaws that is configured for insertion through a working channel of an endoscope catheter. The set of jaws is configured for selective movement between an open position and a closed position. At least one of the jaws defines a cutting edge that is configured for excising tissue when the jaws are moved to the closed position and the jaws defining a substantially enclosed cavity for capturing excised tissue when in the closed position. A jaw locking assembly is configured for selectively actuating the jaws to maintain the jaws in the closed position without relying on positioning the forceps jaws within the working channel. A pull cable assembly is configured for operating the jaw locking assembly to selectively actuate the jaws between the closed position and the open position.

In another embodiment, a method is disclosed for a correcting tissue sheath interference disorder in an anatomical joint. A hypodermic needle and catheter are inserted into tissue near the joint. The needle and catheter both having distal and proximal ends and the catheter having a lumen and the needle extending through the catheter lumen such that the distal end of the needle extends past the distal end of the catheter. The distal end of the needle includes a cutting edge for puncturing tissue. The needle and catheter are inserted into the tissue near the joint using the cutting edge to puncture the tissue while guiding the catheter to position the distal end of the catheter near the tissue sheath. The needle is removed from the tissue and from the catheter while maintaining the catheter in the tissue near the joint as well as maintaining the distal end of the catheter positioned in the tissue near the tissue sheath. A distal end of an endoscope probe is inserted into the lumen at the proximal end of the catheter. The distal end of the probe is guided through the lumen to the tissue sheath near the distal end of the catheter. The tissue sheath is imaged with the probe to determine the position of the probe relative to the tissue sheath. The probe is moved longitudinally in the catheter lumen to extend the distal end of the probe from the distal end of the catheter to interpose the probe between the tissue sheath and an associated anatomical structure. A cutting tool is extended from the distal end of the probe to the tissue sheath. The probe is pulled to move the distal end of the probe and the cutting tool toward the distal end of the catheter such that the cutting tool cuts the tissue sheath. The probe and catheter are removed.

In addition to the exemplary aspects and embodiments described above, further aspects and embodiments will become apparent by reference to the drawings and by study of the following descriptions.

### BRIEF DESCRIPTION OF THE DRAWINGS

FIG. 1 is a diagrammatic illustration of an endoscope system.

FIG. 2a is a diagrammatic illustration of an end face of an imaging fiber showing multiple individual fiber cores.

FIG. 2b is a diagrammatic illustration of a spatially consistent imaging fiber.

FIG. 3 is a diagrammatic illustration of a spatially inconsistent imaging fiber.

FIG. 4 is a diagrammatic illustration of an endoscope with handle and a round biopsy tool in a round working channel.

FIG. 5 is a diagrammatic illustration of an end face of an endoscope having a reniform working channel.

FIG. 6 is a diagrammatic illustration of an illumination profile created by two illumination fibers adjacent to the reniform working channel.

FIG. 7 is a diagrammatic illustration of a cutting tool which is integrated into the reniform working channel of the endoscope.

FIG. 8 is a partial cutaway diagrammatic illustration of the reniform working channel revealing the cutting tool.

FIG. 9 is a diagrammatic illustration of reniform shaped forceps that are integrated into the reniform working channel.

FIG. 10a is a diagrammatic illustration of forceps in one orientation and the requirement that rotation be possible in order to orient with the sample of interest.

FIG. 10b is a diagrammatic illustration of the forceps shown in FIG. 10a in another orientation and the requirement that rotation be possible in order to orient with the sample of interest.

FIG. 11 is a diagrammatic illustration of an endoscope handle that has integrated rotation capabilities.

FIG. 12 is a diagrammatic illustration, in perspective, of an endoscope handle that allows access to the reniform working channel.

FIGS. 13a-13c are a diagrammatic cut away illustrations of the endoscopic handle that illustrates how the fiber is twisted around contours in a cavity that protects the axially located working channel.

FIG. 14 is a diagrammatic perspective illustration of an endoscope handle with a removable actuator for tool actuation, rotation and bending that fits in the reniform working channel of a probe.

FIG. 15 is a diagrammatic exploded perspective illustration of the endoscope shown in FIG. 14.

FIG. 16 is a diagrammatic cut away illustration of the removable actuator for tool actuation, rotation and bending assembly that fits into the reniform working channel.

FIGS. 17a-17b are diagrammatic illustrations of the removable actuation, rotation and bending assembly for the endoscope illustrating operation of the forceps.

FIGS. 18a-18b are diagrammatic illustrations of the removable actuation, rotation and bending assembly for the endoscope illustrating operation of bending.

FIGS. 19a-19b are diagrammatic illustrations of the removable actuation, rotation and bending assembly for the endoscope illustrating operation of bending and operating the forceps.

FIG. 20 diagrammatic cut away illustration of the tool actuation and rotation control.

FIG. 21 diagrammatic cut away illustration of the tool actuation and rotation control operating the forceps.

FIG. 22a is a diagrammatic top view illustration of the endoscope shown in FIG. 14.

FIG. 22b is a diagrammatic cut away illustration of the tool actuation and rotation control illustrating the forceps locking feature.

FIGS. 23a-23b are diagrammatic cut away illustrations of the tool actuation and rotation control illustrating the forceps locking and unlocking feature.

FIGS. 24a-24b are diagrammatic cut away illustrations of the trigger actuation and the bending control.

FIGS. 25a-25b are diagrammatic illustrations how rotation and bending can result in steering.

FIGS. 26a-26c are diagrammatic illustrations of how the tool actuation, rotation and bending assembly can be removed from the endoscope.

FIG. 27 is a diagrammatic perspective exploded view of another endoscope having bending and tool actuation control.

FIGS. 28a-28c are diagrammatic cut away illustrations of the trigger actuation that is employed to expose a blade and then bend the blade out of line with the endoscope.

FIGS. 29a-29c are diagrammatic cut away illustrations of the trigger actuation that is employed to expose an electrosurgical electrode and then bend the electrode out of line with the endoscope.

FIG. 30 is a method diagram for correcting a tissue sheath interference disorder in an anatomical joint.

FIGS. 31a-31f are diagrammatic illustrations of the tissue sheath interference procedure performed on a finger.

FIGS. 32a-32g are diagrammatic illustrations of the tissue sheath interference procedure performed on a wrist.

FIG. 33 is a diagrammatic illustration of the tissue sheath interference procedure performed on a finger using electrosurgery.

FIG. 34 is a diagrammatic illustration of the tissue sheath interference procedure performed on a wrist using electrosurgery.

FIG. 35 is a diagrammatic illustration of the tissue sheath interference procedure performed on a foot using electrosurgery.

FIG. 36 is a diagrammatic illustration of the tissue sheath interference procedure performed to correct Morton's neuroma.

FIG. 37 is a diagrammatic illustration of the tissue sheath interference procedure performed to correct plantar fasciitis.

FIG. 38 is a diagrammatic illustration of an endoscope procedure performed on a joint of a patient.

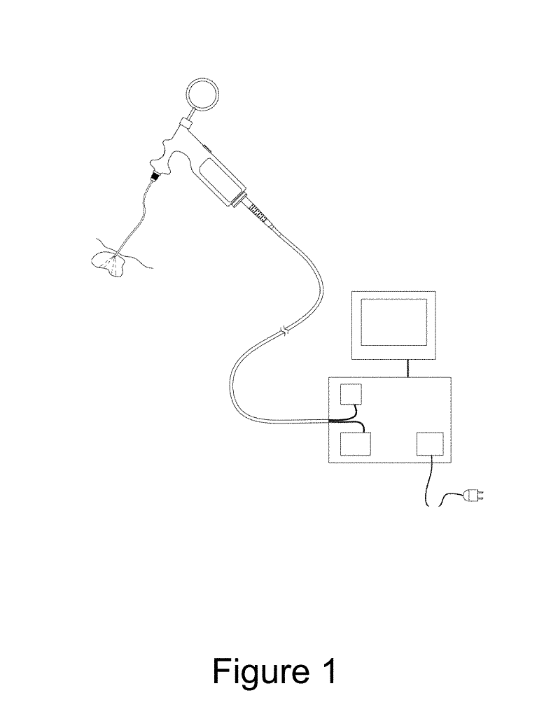

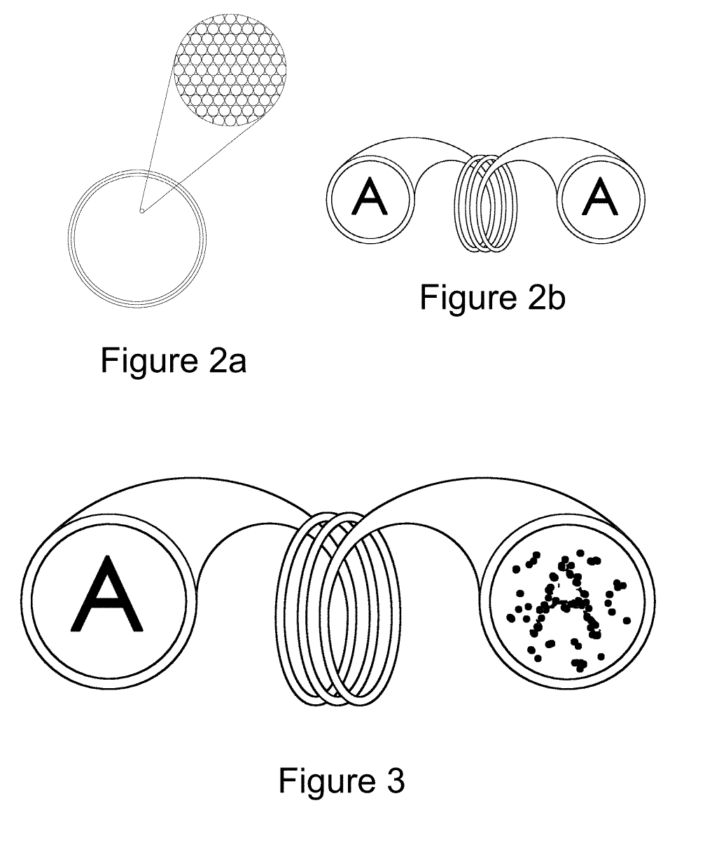

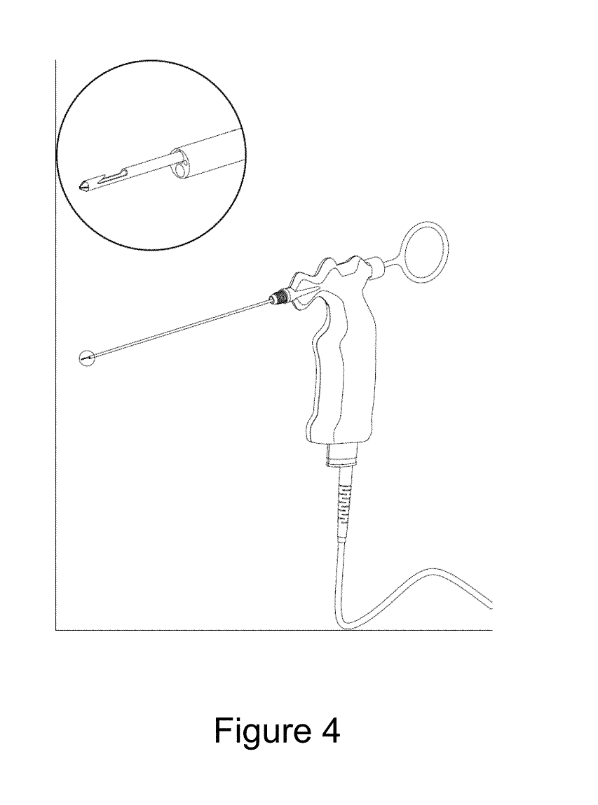

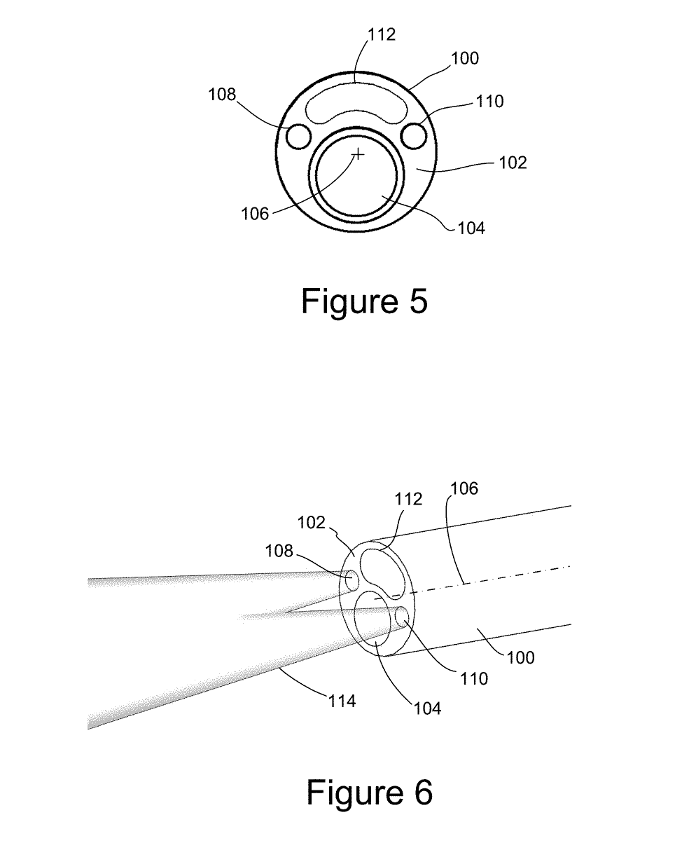

### DETAILED DESCRIPTION

The following description is presented to enable one of ordinary skill in the art to make and use the invention and is provided in the context of a patent application and its requirements. Various modifications to the described embodiments will be readily apparent to those skilled in the art and the generic principles taught herein may be applied to other embodiments. Thus, the present invention is not intended to be limited to the embodiments shown, but is to be accorded the widest scope consistent with the principles and features described herein including modifications and equivalents, as defined within the scope of the appended claims. It is noted that the drawings are not to scale and are diagrammatic in nature in a way that is thought to best illustrate features of interest. Like items may refer to like components throughout the various views of the Figures. Descriptive terminology may be adopted for purposes of enhancing the reader's understanding, with respect to the various views provided in the Figures, and is in no way intended as being limiting.

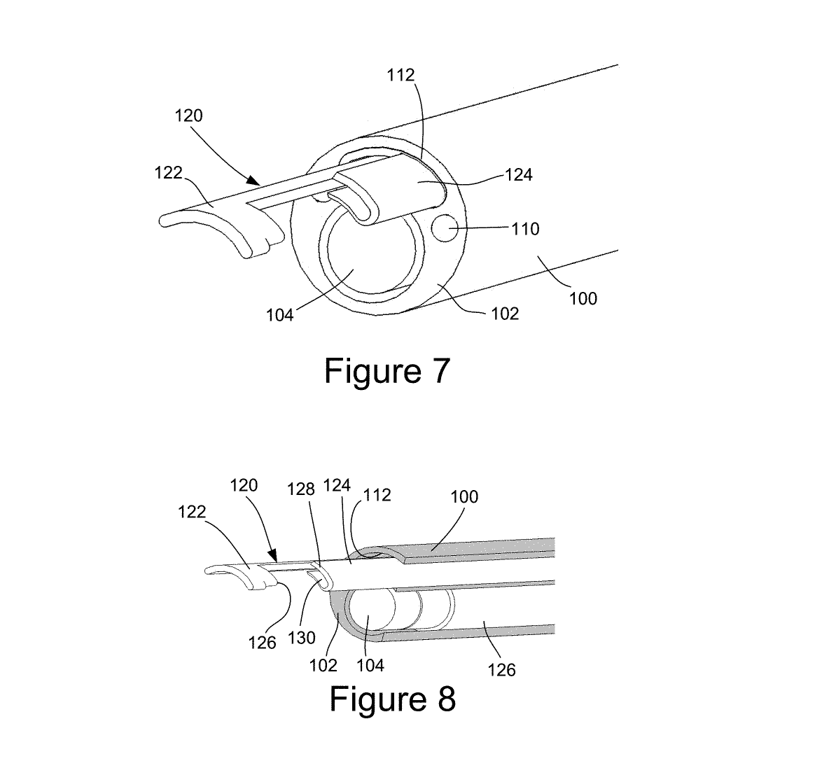

Referring now to FIG. 5, an embodiment of an endoscope probe 100 is shown having a distal end 102 and an objective lens 104 that is positioned at the distal end for imaging a field of view into a distal end of an imaging fiber bundle. The objective lens and imaging fiber bundle have a large enough cross-sectional area that they encompass a longitudinal center axis 106 of the probe. The endoscopic probe also includes two illumination fibers 108 and 110. When the diameter of the endoscope probe is reduced to sub-millimeter proportions, employing as much of the available cross sectional area becomes critical. For instance, while a round (e.g., circular) working channel is fairly common for guiding and supporting tools, it does not fully utilize the possible real estate. An embodiment of a reniform shaped working channel 112, however, can maximize the available cross-sectional use while limiting the effect on imaging capability. In other words, and for instance, to achieve the same cross-sectional area as a reniform shaped working channel in a 1 mm diameter endoscope, the lateral extent of the imaging optics at the distal end of the endoscope would have to be decreased by over thirty percent which would significantly degrade the image quality.

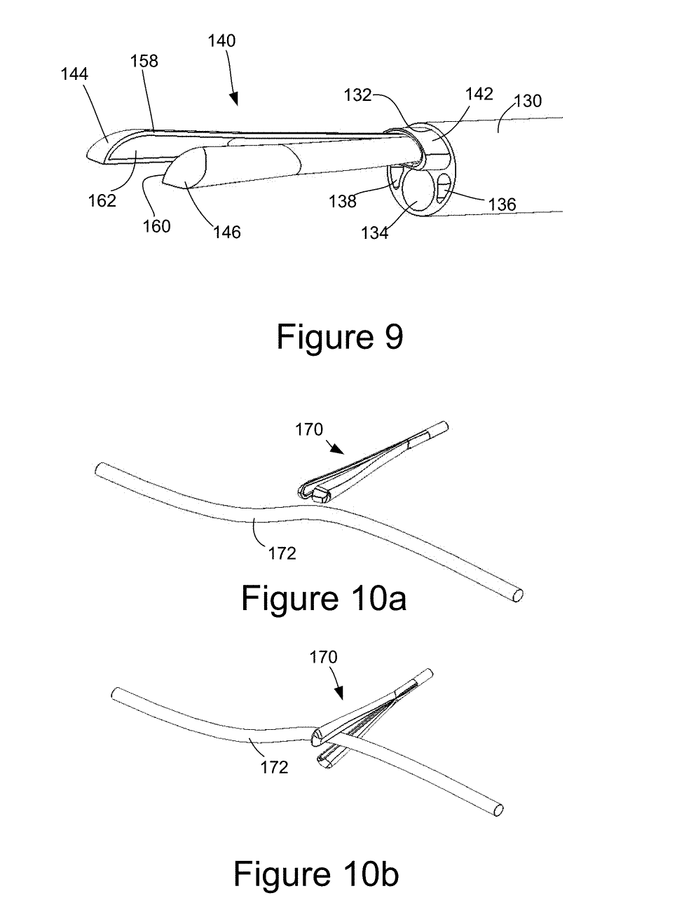

Referring now to FIG. 6 in conjunction with FIG. 5, in order to obtain an image from a field of view of an image area inside of a cavity, it is necessary that the endoscope provide illumination light 114 at the distal end of the probe. Therefore, along with the imaging fiber, one or more simple multi-mode fibers, such as illumination fibers 108 and 110, are employed to transmit light to the distal end of the endoscope and provide illumination to the imaging area. Unlike the imaging fiber, in some instances, the illumination fibers do not require lenses but can instead rely upon the cone of light that the illumination fibers emit to fill the field of view of the imaging lens. Generally, fibers with a large numeric aperture can be utilized for illumination as this increases the field of illumination and increases the uniformity.

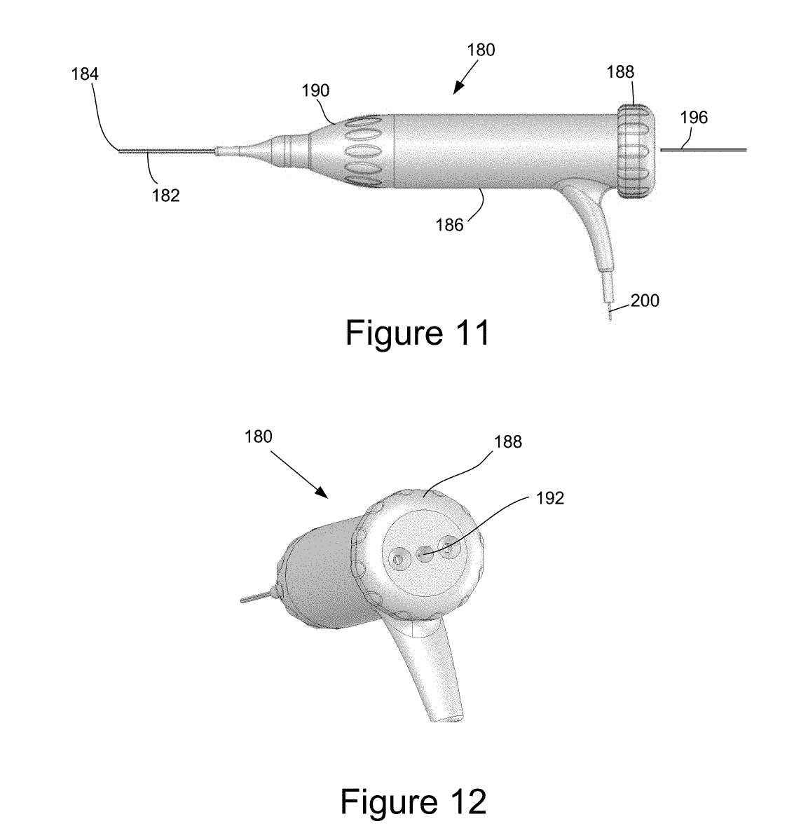

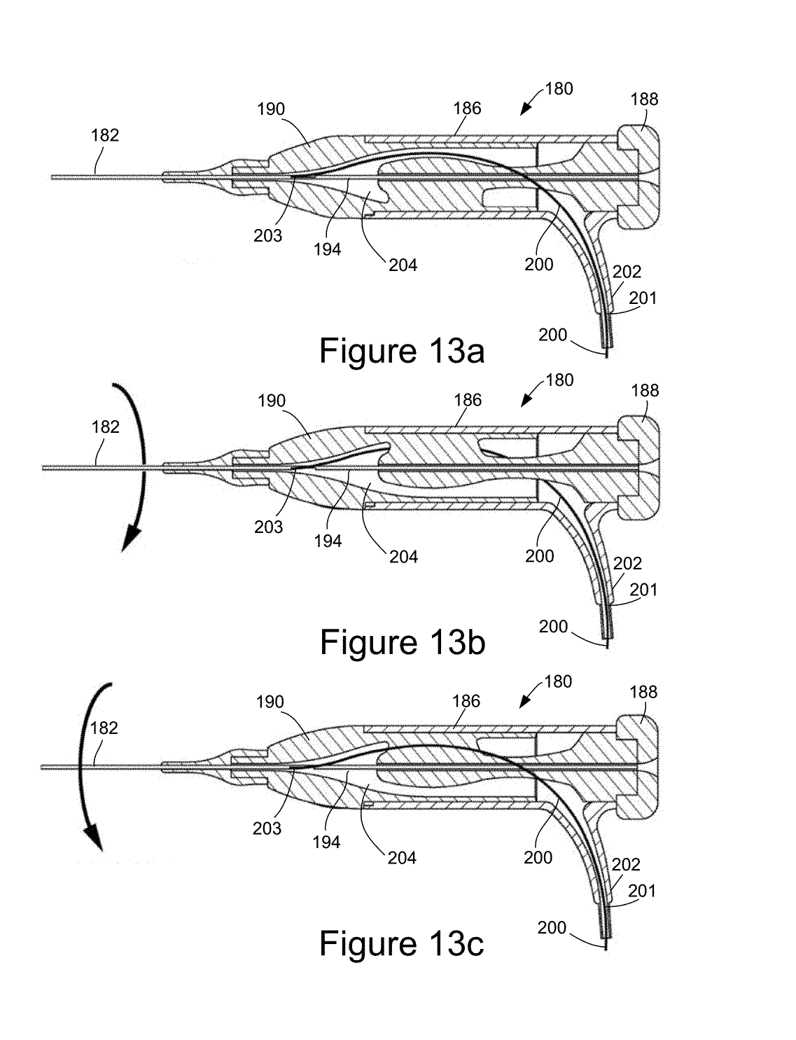

Referring now to FIGS. 7 and 8 in conjunction with FIGS. 5 and 6, a tool 120 is configured for use with the reniform working channel. The tool is sized and shaped to be guided to the distal end of the probe using the reniform working channel. To efficiently utilize the cross-sectional space available, the tool can be configured such that the probe having the reniform working channel plays a mechanical role as well as a guidance role. For instance, tool 120 can be a cutter or biopsy tool, as illustrated in FIG. 7, which includes an arm structure 122 and a blade structure 124 that can be moved longitudinally relative to one another to cut tissue between a surface 126 and a blade edge 128 and samples can be captured in a cavity 130. The arm and blade structures can be held in operable communication with each other laterally during actuation by the working channel. Applicants recognize that the disposable nature of this device, and therefore the single use application, lends itself to a tool that is not completely autonomous in operation without the probe but instead relies on the nature of the working channel to hold the two portions of the tool together. FIG. 8 shows a cutaway portion of the probe and the working channel in which the structures of tool 124 operatively engage opposite walls to maintain a fixed lateral relative position to one another. FIG. 8 also shows an imaging fiber 126.

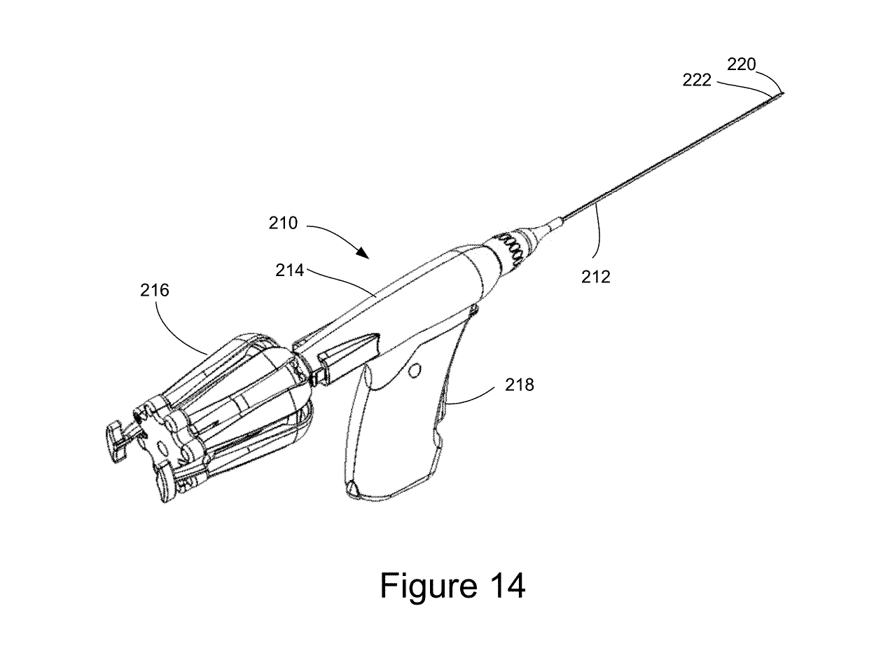

Referring now to FIG. 9, an embodiment of an endoscopic probe 140 includes an imaging lens 134 and illumination fibers 136 and 138. Probe 140 includes a reniform working channel 132 that can incorporate materials, other than just the material from which the probe is formed, which each can play a role in the operation of the tool. For example, in an embodiment, a reniform shaped forceps tool 140 can require the working channel to include a metal sleeve 142 which closes forceps jaws 144 and 146 of the forceps more readily than would a non-metal material when the forceps jaws are moved longitudinally to retract into the reniform working channel. As shown in FIG. 9, the forceps jaws can include cutting edges 158 and 160 which can cut tissue and can be formed to define a cavity 162 for capturing tissue. The reniform working channel inclusive of the tool also increases the torsional rigidity (limits twisting) of the entire endoscope whereas a circular working channel and tool are less effective for this purpose.

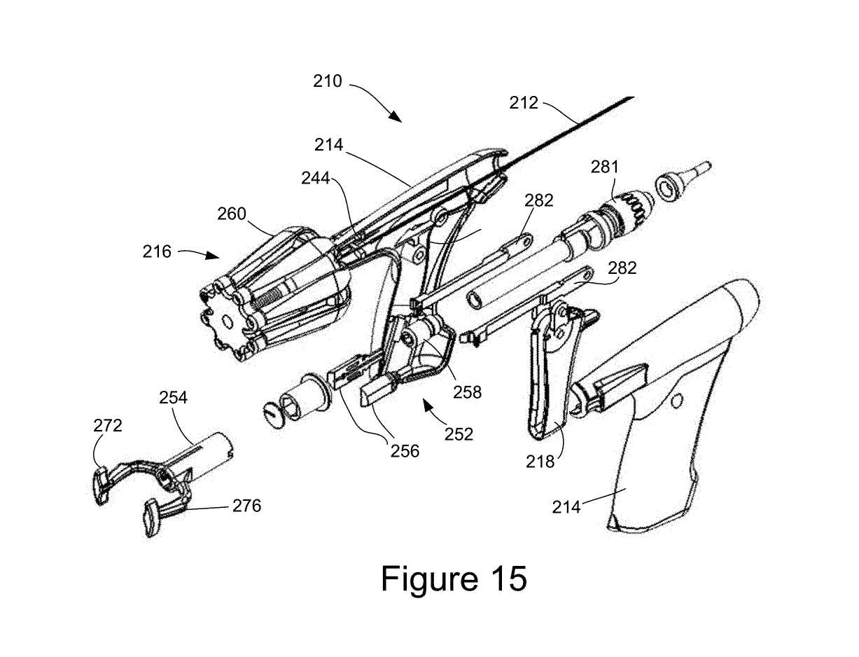

The use of the reniform working channel does impose a constraint on the positioning of the tool relative to the tissue of interest. This is significantly beneficial for purposes of removing a tool from the working channel and inserting a new tool. The reniform shape of the working channel insures that the new tool is oriented the same way that the removed tool was oriented. Referring now to FIGS. 10a and 10b, when a tool 170 does require rotation, for instance, in the case when the forceps require rotation from a first oriented (FIG. 10a) to a second orientation (FIG. 10b), that is perpendicular to the first orientation, in order to grab a suture or cut a suture 172, the entire distal end of the endoscope may be rotated.

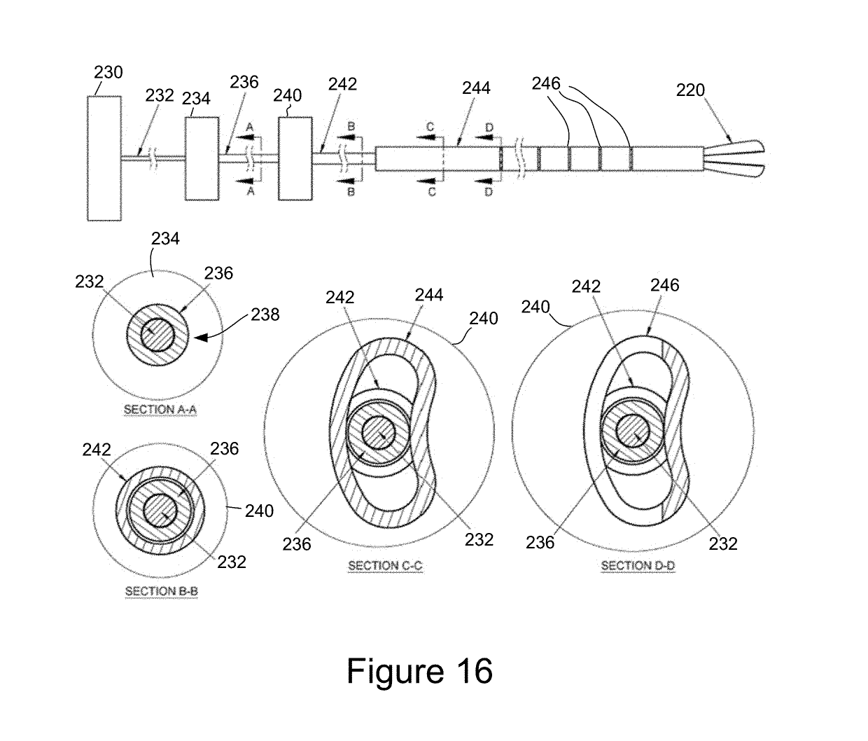

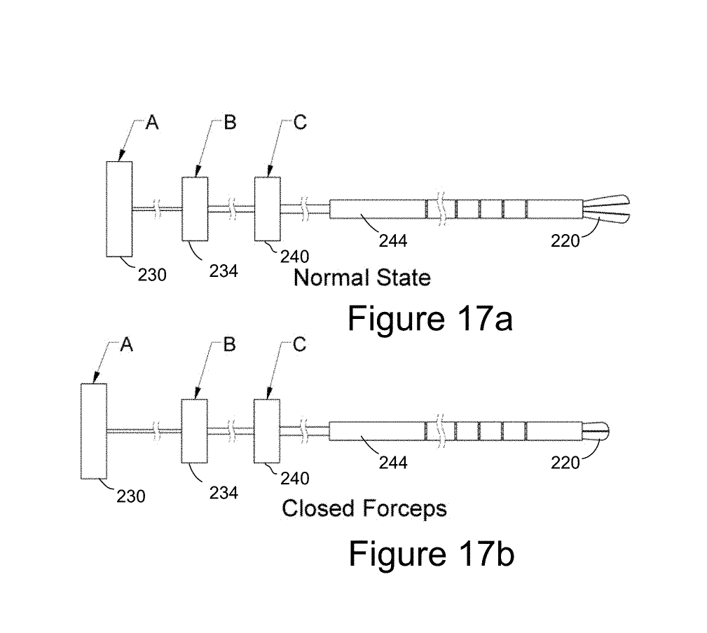

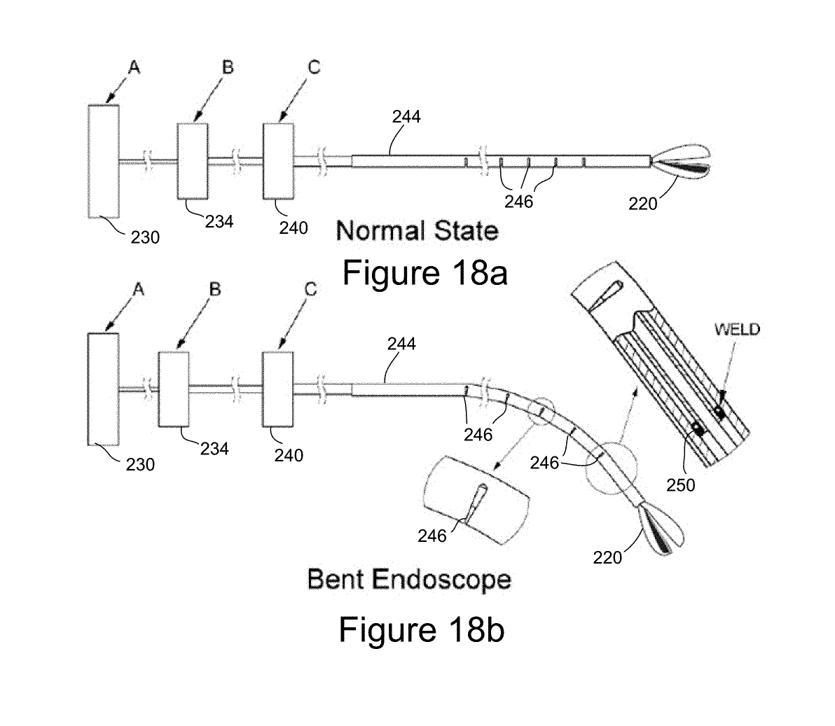

Referring collectively to FIGS. 11, 12 and 13a-13c, an embodiment of an endoscope 180 is shown which includes an endoscope handle body 186 connected to a proximal end of a probe 182. The probe includes a distal end 184 which can be configured similar to the distal probe and shown in FIG. 5. The probe can be of any suitable length. The endoscope includes a knob 188 and a cone 190 supported by handle body 186. A proximal end of the probe (FIGS. 13a-13c) is received through the cone such that the knob and cone can be rotated relative to the body to rotate the probe. The endoscope handle body includes a port 192 (FIG. 12) in the knob which accesses a proximal end of a working channel 194 (FIG. 13a) for the insertion of endoscopic tools, such as a tool 196, into the working channel.

Referring now to FIG. 13a-13c in conjunction with FIGS. 11, 12, the former are elevational cut-away views illustrating further details of the embodiment of endoscope 180. An imaging fiber 200 is attached to handle body 186 at connection location 201 in a handle member 202 and is connected to a proximal end 203 of the probe near cone 190. Handle body 186 defines a cavity 204 which houses or receives the imaging fiber between the handle member and the cone. When the knob and/or cone are rotated relative to the handle body, the imaging fiber is twisted and can rotate inside the cavity around the working channel while still allowing tool access to the working channel. As shown in FIG. 13a, when the knob is at a centered position relative to the handle body, the imaging fiber is not twisted as it passes through the cavity. When the knob is rotated, for example, 177° clockwise, as shown in FIG. 13b, the imaging fiber is twisted 177° clockwise in the cavity. When the knob is rotated, for example, 177° counterclockwise, as shown in FIG. 13c, the imaging fiber is twisted 177° counterclockwise in the cavity. Although the illumination fibers are not shown, these fibers can be arranged similarly to the imaging fiber. The internal structure of the handle body allows the imaging and illumination fibers to be twisted without breaking. While it is difficult to twist a larger diameter imaging fiber, the sub-millimeter diameter imaging fiber that is employed in embodiments of the scope described herein, may be twisted fairly easily. This allows the manufacture of an endoscope to allow rotation of the entire endoscope by allowing the imaging fiber to undergo a near 360 degree twisting motion. Further, because the endoscope being described is disposable in nature, the effect of repeated twisting of the imaging fiber on the lifetime of the imaging fiber is not important. It should be appreciated that the rotational ranges described herein are not intended as being limiting and any suitable range can be used while still employing the teachings herein.

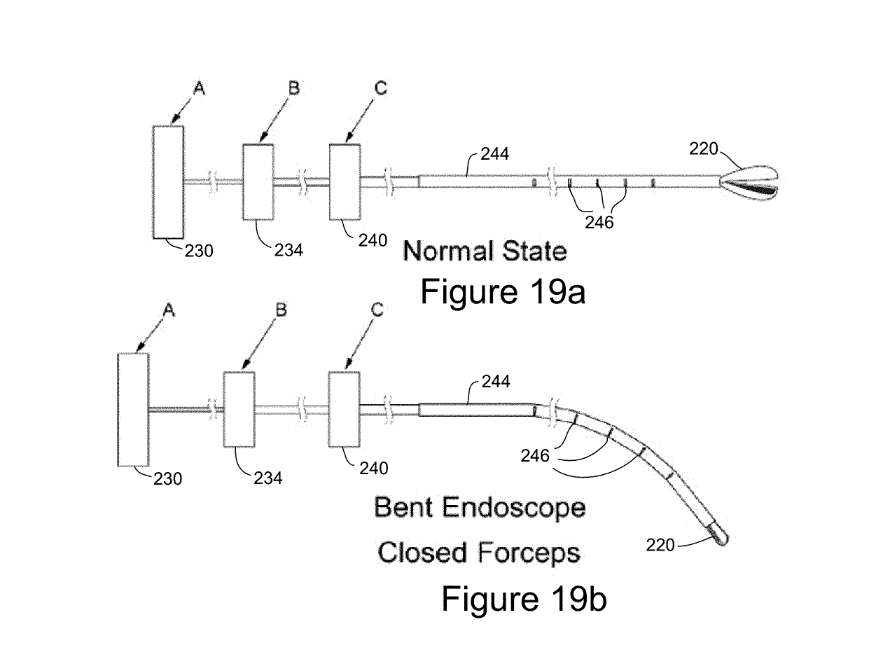

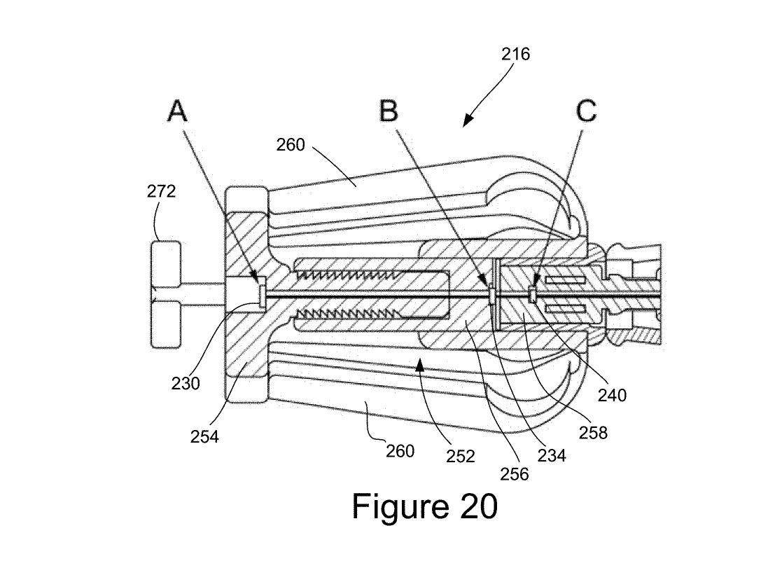

Referring now to FIGS. 14 and 15, an embodiment of an endoscope 210 is shown in perspective and exploded perspective views, respectively, which provides for rotation and bending of the endoscope to allow control of the position and path of the endoscope during insertion and direction to the tissue of interest. Also, while embodiments of the endoscope can include a working channel having a reniform shape, this is not required for many of the different functions illustrated. Endoscope 210 includes a probe 212, a handle body 214, an actuator 216, and a trigger arrangement 218. Actuator 216 is connected to the probe and can rotate the probe relative to the handle body. Endoscope 210 also includes a front cone 281 which can be used for rotating the probe and an optical fiber 283 for illumination and/or imaging. A tool head 220 is shown extending from a distal end 222 of the probe.

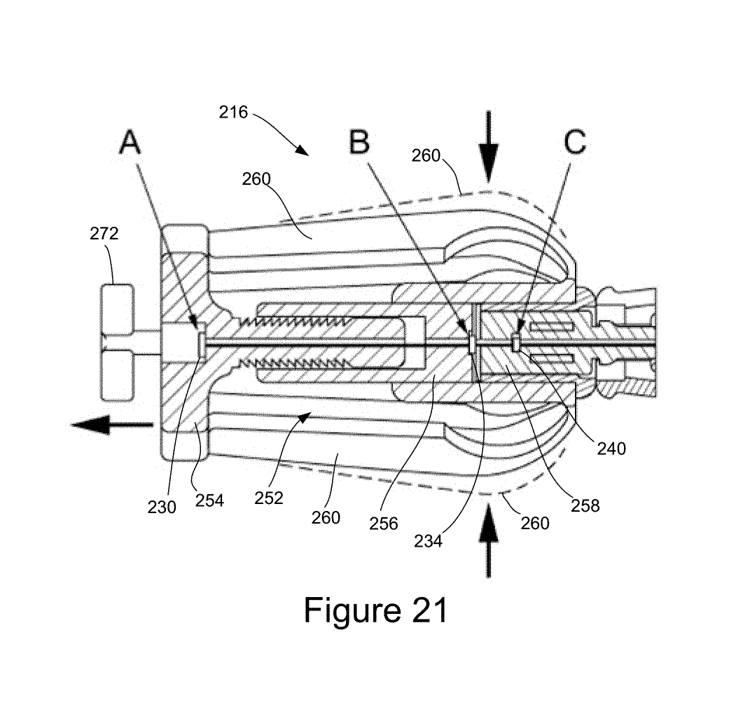

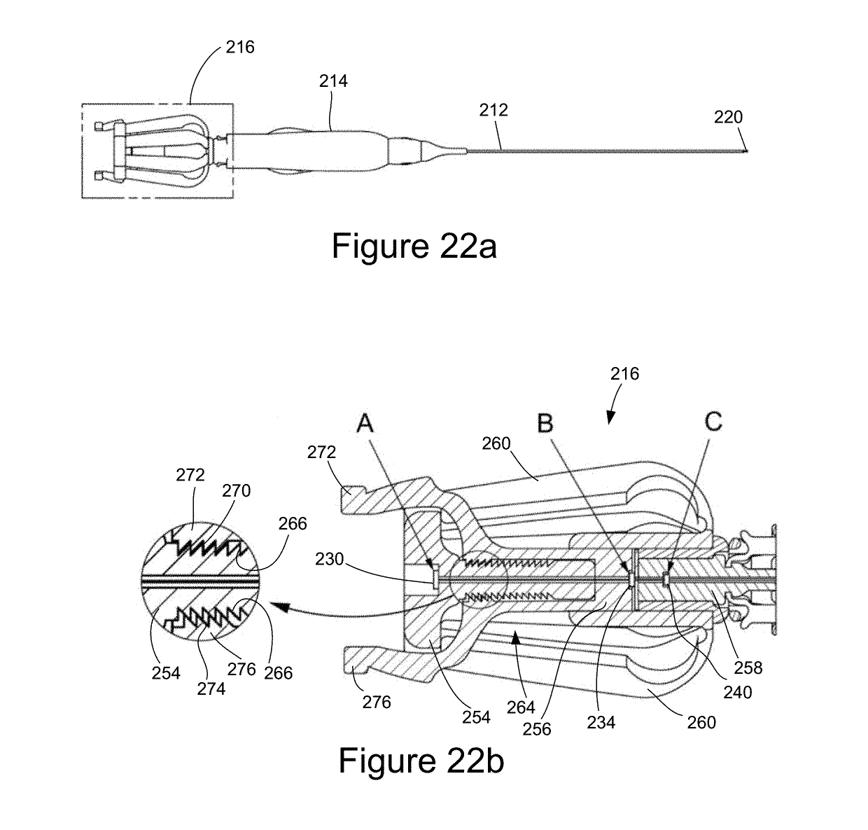

Referring now to FIGS. 16 through 19 in conjunction with FIG. 14, attention is now directed to the internal series of concentric wires and tubes that allow the endoscope to be operated. FIG. 16 is a diagrammatic cut-away view of the handle and endoscope which reveals a wire and tube assembly including four specific components. The components and their physical relationships are illustrated by FIGS. 17-19. Working from left to right (FIG. 16), a first disk 230 (or “disk A”) is attached to a single wire 232. This wire passes through three different hollow structures before being attached to tool head 220 (forceps jaws). The wire passes through a second disk 234 (“disk B’) which is itself attached to a fine tube 236 through which wire 232 moves freely, see section A-A. A friction reducing liner or lubricant can be used to insure the two pieces do not bind. Again, the disposable nature of the assembly allows the necessity that the components can be disassembled for cleaning and common biologically acceptable lubricants may be employed without concern of cross contamination. A mutual assembly 238 of tube 236 and wire 232 pass through a third disk 240 (“disk C”), see section B-B, which itself is attached via a larger tube 242. The latter is attached to an outer sheath 244, see section C-C, which includes kerf cuts 246, see section D-D.

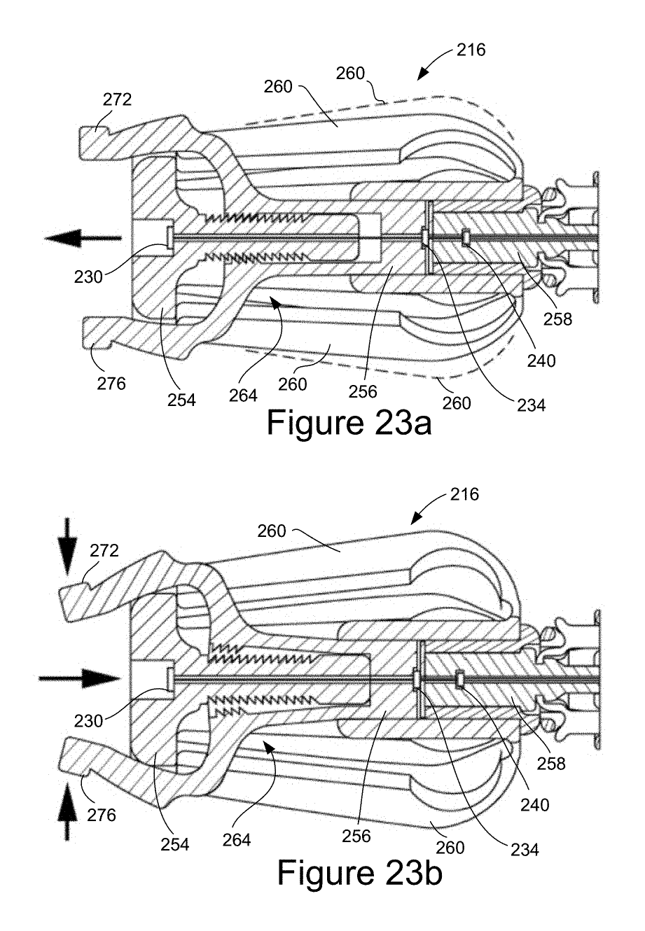

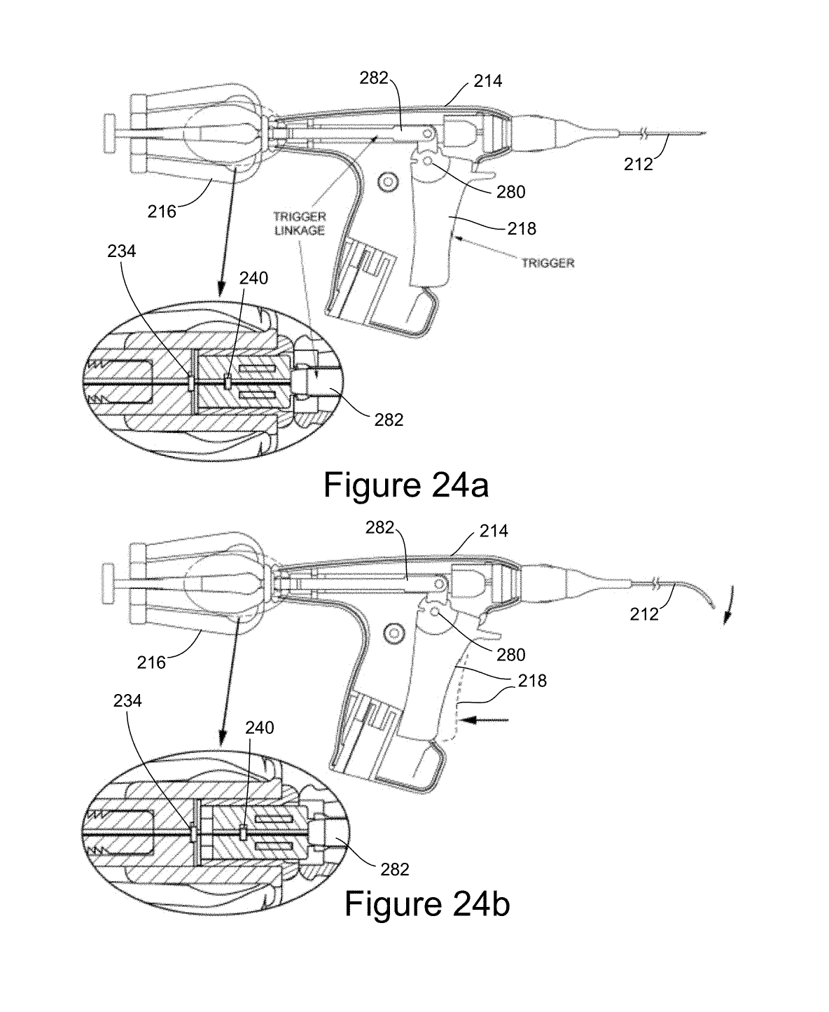

The different functions of the endoscope are controlled independently through the relative positions of disks 230 (A) and 234 (B) with respect to each other and with respect to disk 240 (C). Specifically, as shown in FIGS. 17a and 17b, pulling disk 230 (A) away from disk 240 (C) while leaving disk 234 (B) in place will close forceps jaws 220, as can be seen by a comparison of FIGS. 17a and 17b. As shown by comparing FIGS. 18a and 18b, holding disk 230 (A) in place and moving disk 234 (B) away from disk 240 (C) will pull on tube 236, that is connected by a weld 250 (see inset in FIG. 18b) to the outer sheath 244 just past a series of the kerf cuts 246 in the outer sheath to cause the endoscope to bend. It should be noted that regardless of the position of disk 234 (B) relative to disk 240 (C), movement of disk B will not affect the position of disk 230 (A). Therefore, bending the endoscope will not alter the closed or open state of the forceps (tool head 220). As shown by comparing FIGS. 19a and 19b, moving disk 230 (A) relative to disk 240 (C), regardless of the position of disk 234 (B), will close the forceps and moving disk 230 (A) and disk 234 (B) relative to disk 240 (C) will bend the probe and close the forceps.

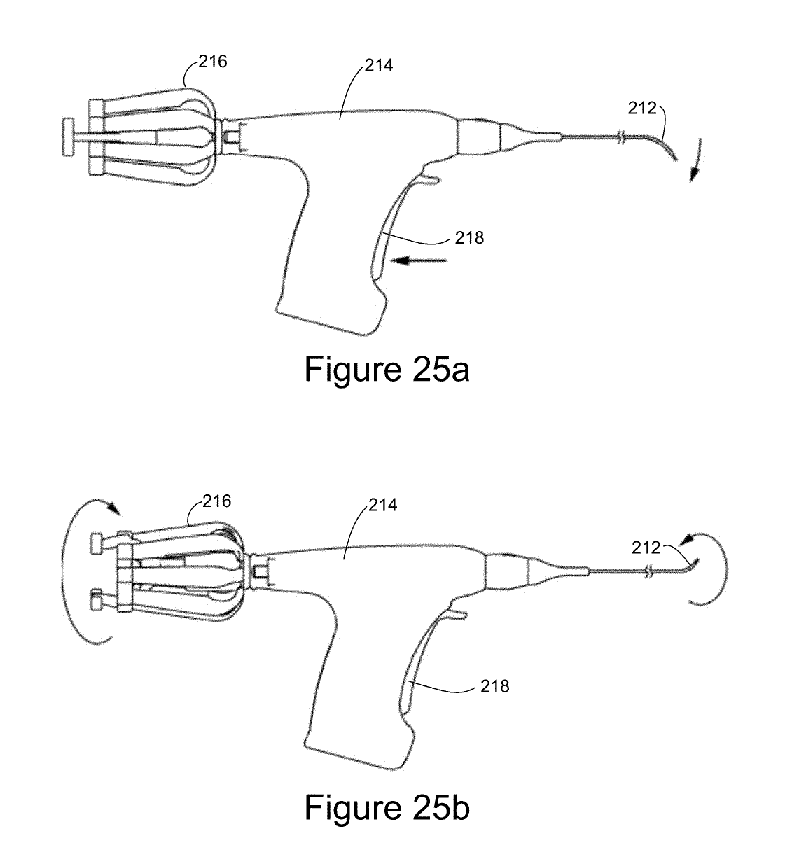

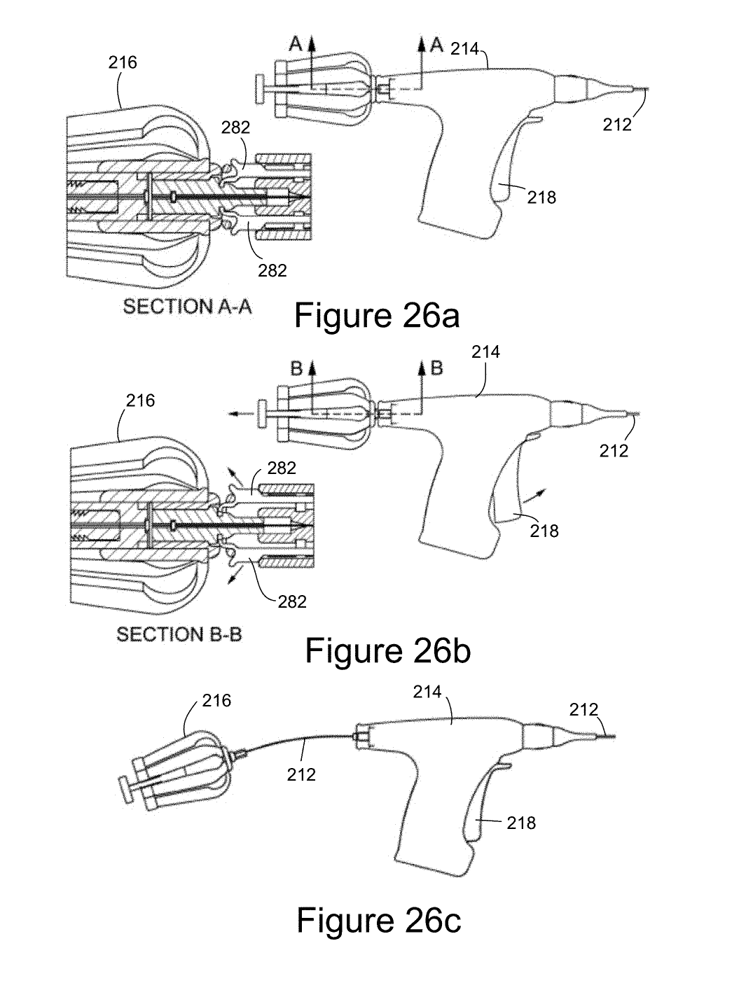

Referring now to FIG. 20 in conjunction with FIGS. 14-19, an embodiment of actuator 216 of endoscope 210 is shown in a partially cut-away view which illustrates structures for moving and maintaining the positional relationships between the disks during use. Actuator 216 is located on the rear of the handle and is referred to colloquially as the “Squid” due to the shape although any suitable shape can be used. The actuator is connected to probe 212 and handle body 214 (FIG. 15) such that rotation of the actuator rotates the probe. FIG. 20 shows a core arrangement 252 having a proximal core section 254 attached to disk 230 (A); a middle core section 256 attached to disk 234 (B); and a distal core section 258 attached to disk 240 (C). As shown in FIG. 21, the actuator includes a shell arrangement 260 that is connected to the proximal core section and is configured to be squeezed to produce collapsible movement 261 toward the core arrangement which proximally moves 263 core section 254 and separates disk 230 (A) from disks 234 (B) and 240 (C) without moving disks 234 (B) and 240 (C) to close forceps jaws 220 (FIGS. 17a and 17b). The shape and the material of construction of the shell arrangement can be chosen to alter the tactile response of the component and can also affect the ratio between the “squeeze” and the forceps “bite.” Such tactile customization is not possible with a knob or trigger mechanism.

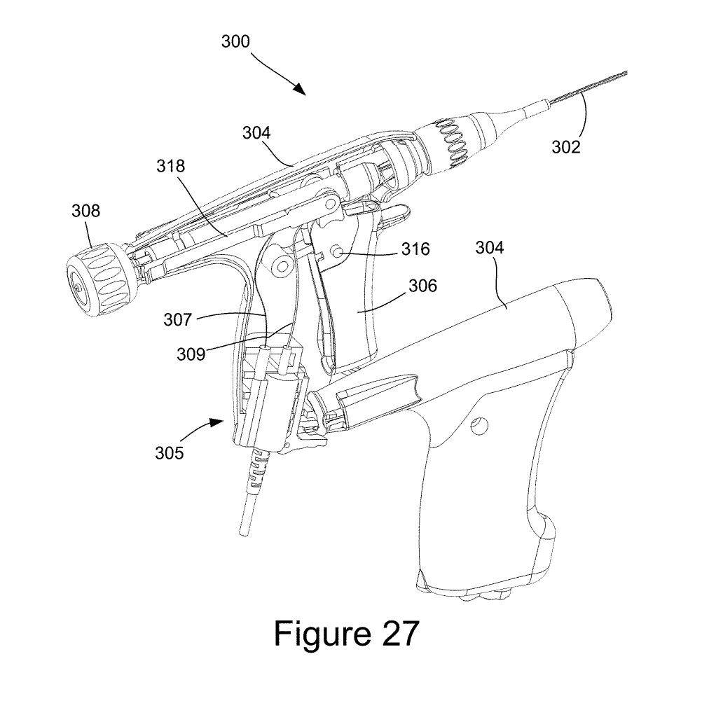

A further feature of an embodiment of actuator 216 is the ability to lock the forceps in a closed position. FIG. 22a illustrates endoscope 210 in a top view, and FIG. 22b shows the actuator in a partial cut-away view that is rotated 90° along the center axis relative to the views shown in FIGS. 20 and 21. An embodiment of locking ratchet mechanism 264 is shown which includes a set of outwardly facing ratchet teeth 266 (see inset) around the entire periphery of core section 254. When the forceps are closed by moving disk 230 proximally by squeezing the shell arrangement to move the core section 254 proximally, outwardly facing ratchet teeth 266 engage inwardly facing ratchet teeth 270 of a latch arm 272 and inwardly facing ratchet teeth 274 of a latch arm 276 on opposite sides of core section 254. These teeth keep the forceps closed even when the bulb of the squid is released back to its original position. An embodiment of the ratchet teeth are arranged such that the engagement of ratchet teeth 266 with teeth 270 is offset by one half of a tooth from the engagement of ratchet teeth 266 with teeth 274 so that the jaws can be locked in positions with a resolution of one half of the distance between the ratchet teeth on either side of core section 254. Put another way, the size and spacing of the teeth of the ratchet mechanism can be limited by current manufacturing techniques and in order to increase the effective resolution of the ratchet step, the teeth of latching arms 272 and 276 can be offset from one another by one half of a tooth so that the latching arms alternate engagement of ratchet teeth 266 on opposing sides of the core section which allows “half steps.”

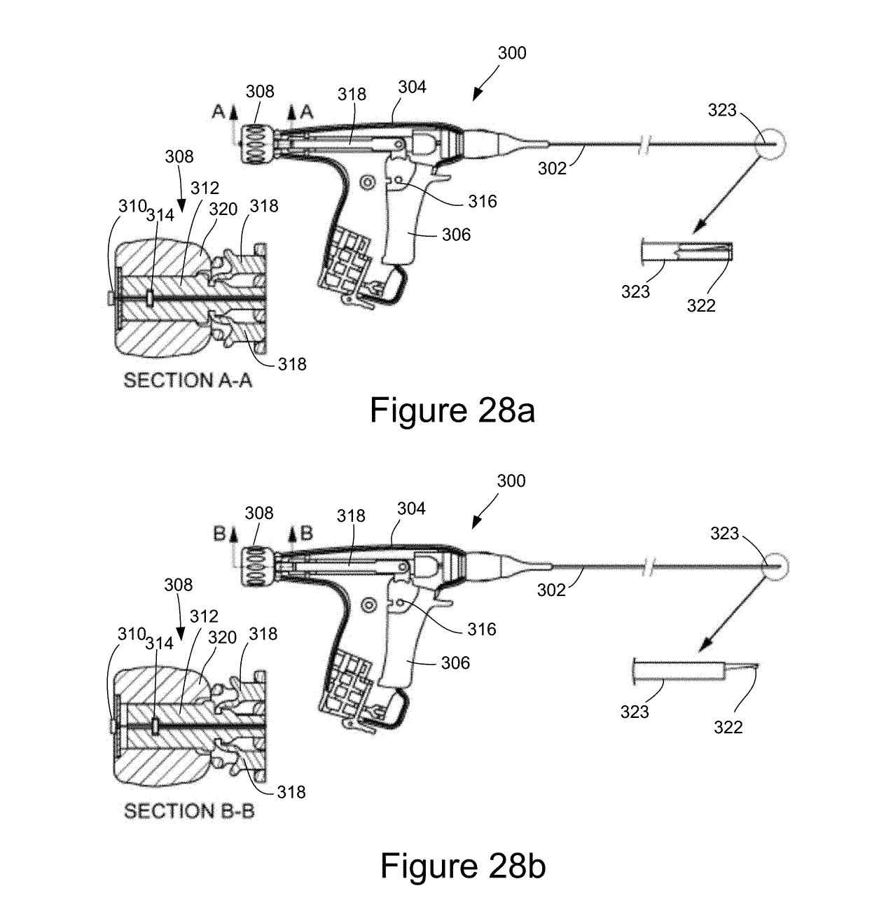

To open the forceps, from a locked position (FIG. 23a) latch arms 272 and 276 are squeezed toward one another as shown in FIG. 23b. This motion distorts the latch arms to pivot against core section 254 and separates the inward facing teeth of the latch arms from the outward facing teeth of core section 254 while simultaneously pushing core section 254 distally which forces forceps 220 to open. The forceps are one of many potential tools that the actuator can operate and should not be construed as the only tool for which the actuator is advantageous as a control device.

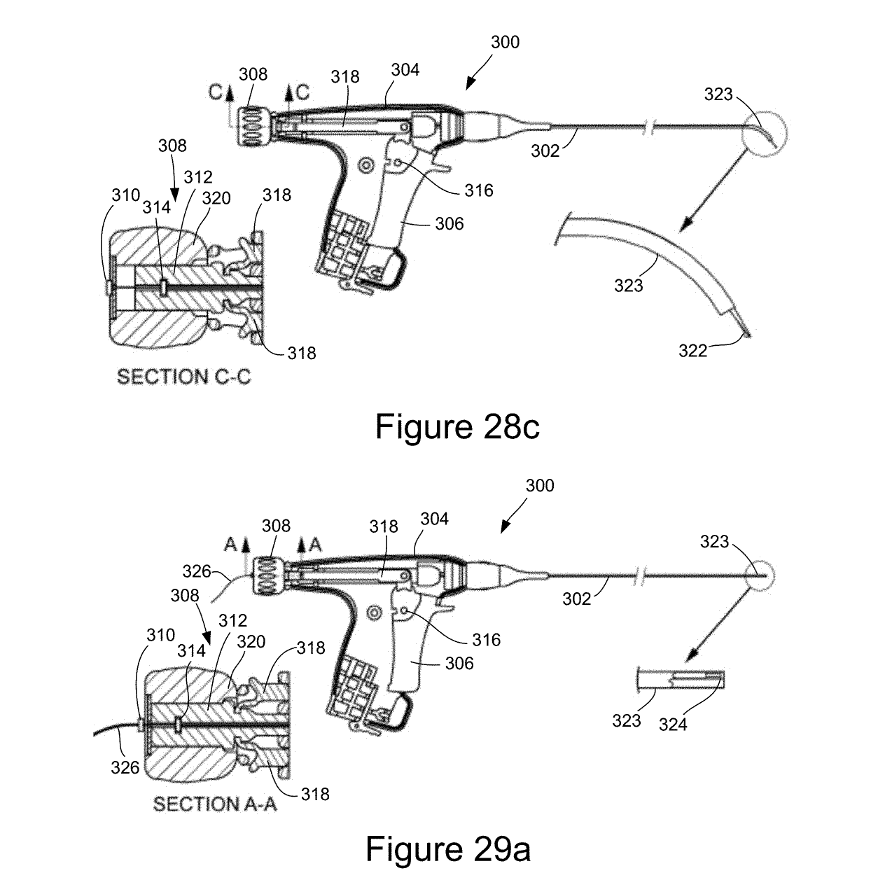

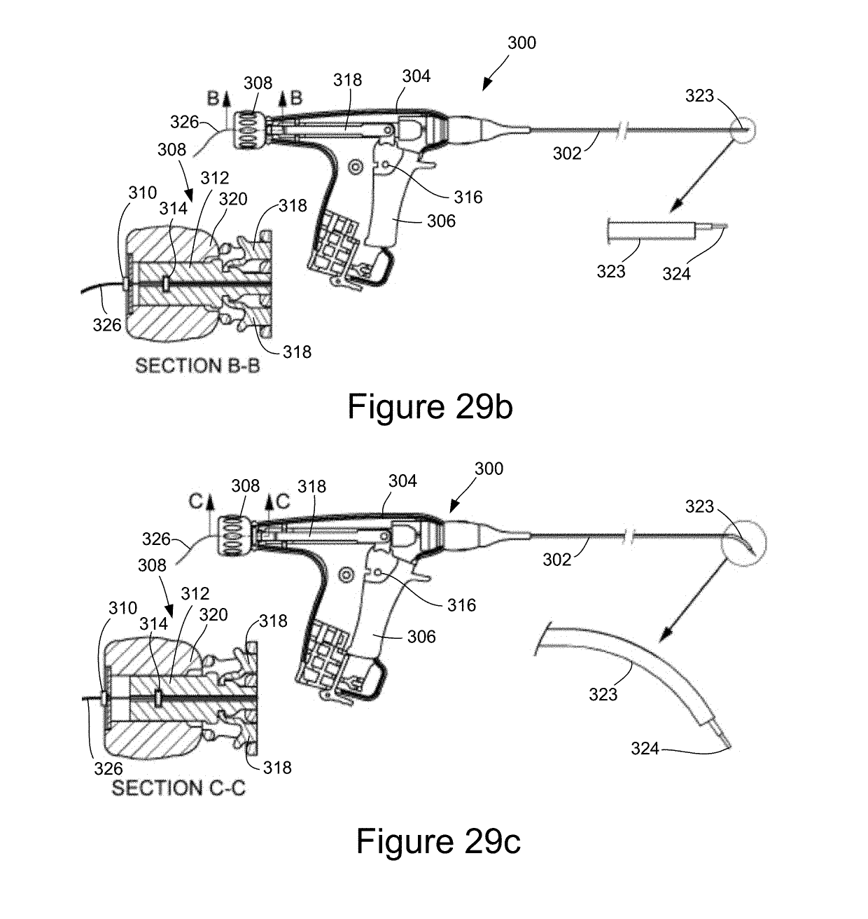

Referring now to FIGS. 24a and 24b in conjunction with FIGS. 16-19, trigger 218 is connected to handle body 214 at a pivot point 280. Depressing the bottom portion of the trigger actuates a linkage 282 which is connected to distal core section 258 to move core section 258 distally and thereby separate disk 240 from disk 234, as can be seen from the inset in FIG. 24a as compared to the inset in FIG. 24b. Moving these two disks relative to each other causes outer sheath 244 in probe 212 to bend along the side with kerf cuts 246 (FIG. 18) which bends the probe. While curving in the downward direction is shown in FIG. 24b relative to FIG. 24a, it should be appreciated that rotating the endoscope probe relative to the handle with the actuator in conjunction with depressing the trigger, as shown by comparing FIG. 25a to FIG. 25b, allows the distal end of the probe to be bent in any radial direction.

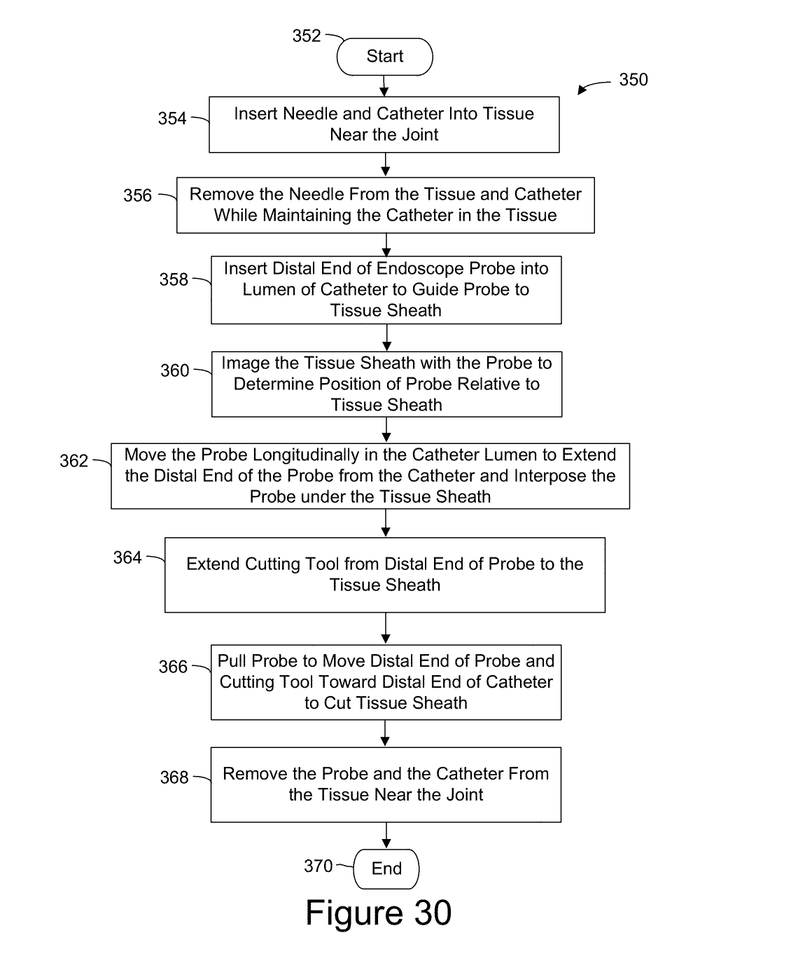

Referring now to FIGS. 26a, 26b and 26c, another feature of endoscope 210 is the ability to collect multiple samples without having to remove the endoscope probe and forceps assembly from a cavity and then reinsert a new endoscope probe and forceps assembly in the cavity. After a sample is collected using endoscope 210, FIG. 26a, the entire steering and forceps mechanism connected to the Squid can be removed from the working channel, FIG. 26c, and a new one inserted. This is accomplished by pressing up on the trigger, FIG. 26b, which unlatches trigger linkages 282 from the actuator, as shown by comparing the inset in FIG. 26a to the inset in FIG. 26b, so that the entire actuator and the related series of disks, wires and tubes can be removed from the handle body and probe while still keeping the forceps locked, FIG. 26c. These features of endoscope 210 improve the ability to bend, rotate and operate the tooling and do so all with a working channel insert that can be removed after completing one task or when the endoscope is to be used for a task that does not require those tools specifically described herein.

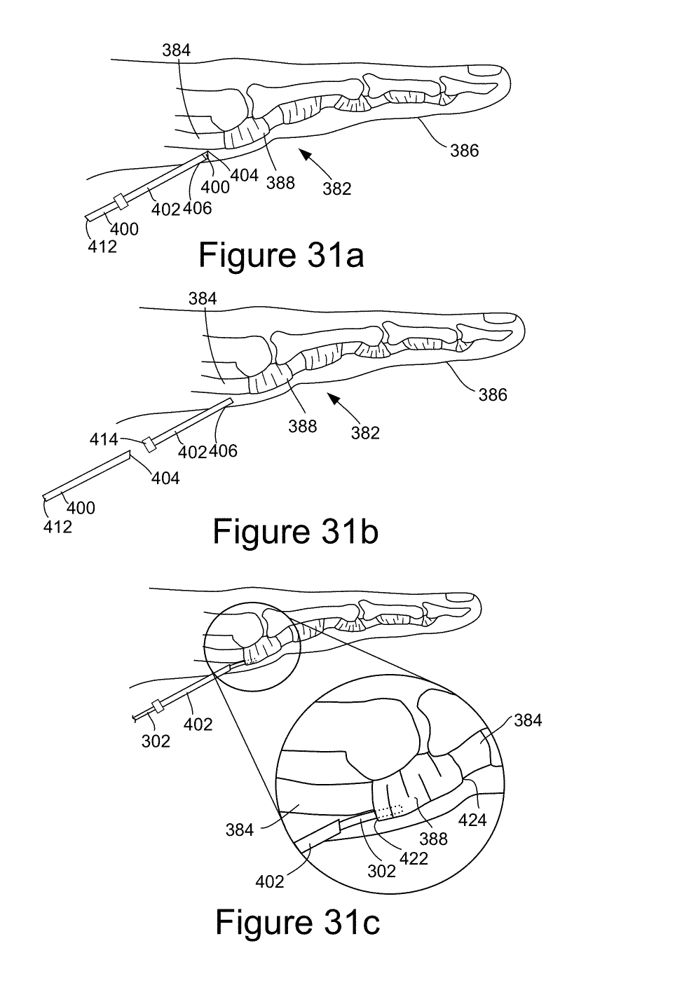

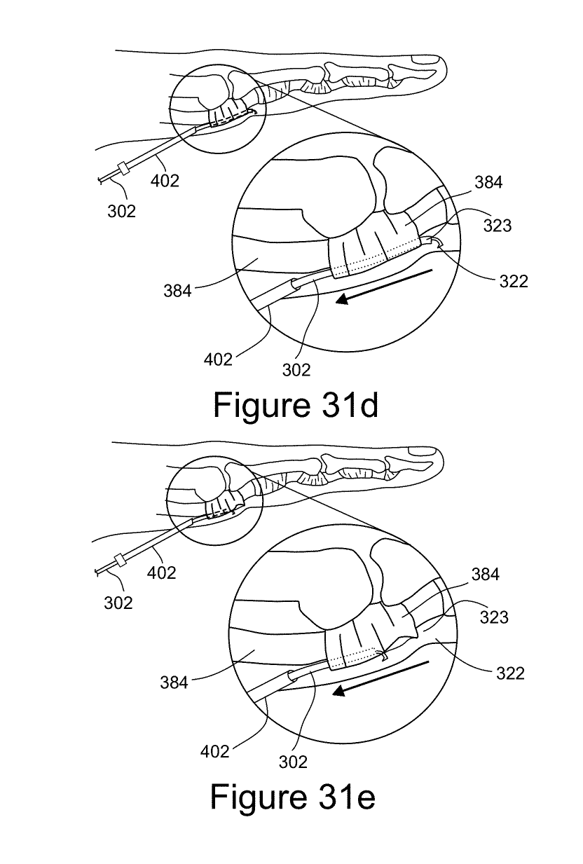

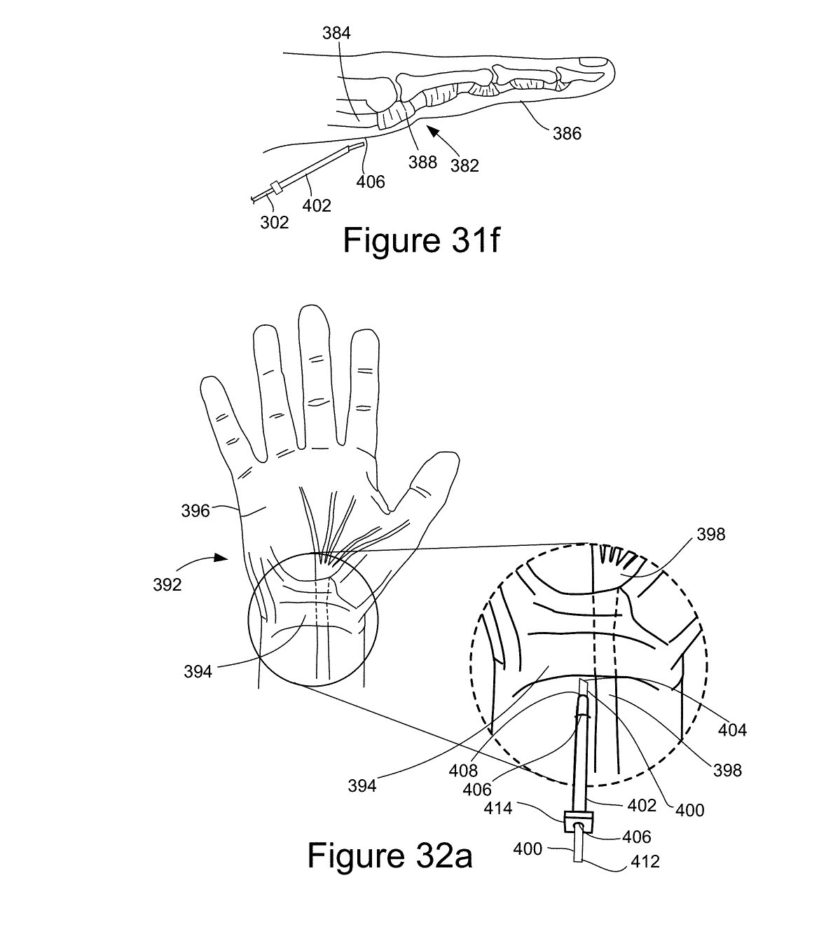

Referring now to FIGS. 27, 28a-28c, and FIG. 29a-29c, an embodiment of endoscope 300 is shown which includes a probe 302, a handle 304, a trigger 306 and an actuator 308. Endoscope 300 also includes an optical assembly 305 which can provide illumination light to an illumination fiber 307 and can receive images from an image fiber 309 for imaging at the distal end of the probe. Actuator 308 includes a knob 320 for rotating instead of the shell arrangement of actuator 216. Cross-sections of actuator 308 in various operative positions are shown inset in each of FIGS. 28a-28c and 29a-29c. Actuator 308 is connected to probe 302 such that rotating the actuator rotates the probe relative to the handle. Actuator 308 includes a disk 310 (B) that is connected to the knob, and a core section 312 that is attached to a disk 314 (C). Trigger 306 is pivotally received by handle 304 at a pivot point 316 and pivotally attaches to actuator linkage 318 which, in turn, connects to actuator 308. In the embodiment shown in FIGS. 28a-28c, a blade 322 is integrated onto a distal end 323 of the probe, (as shown in the insets of the Figures); and in the embodiment shown in FIGS. 29a-29c, an electrosurgical electrode 324 is integrated into distal end 323 of the probe, (as shown in the insets of the Figures) and an electrode power cable 326 extends from the electrode through the probe and the actuator and out to an electrosurgical generator (not shown). The electrosurgical electrode can be a sharp end of a wire or an edge of a flat surface of a conductive material or any other suitable electrically conductive shape. In the at-rest condition, FIGS. 28a and 29a, the blade/electrode is retracted into the endoscopic sheath of probe 302. Depressing the trigger halfway pushes both disk 310 (B) and disk 314 (C) forward which results in the blade/electrode being pushed from the sheath, as can be seen by comparing FIGS. 28a to 28b and particularly by comparing section A-A to section B-B in the insets in FIGS. 28a and 28b, respectively; and as also can be seen by comparing section A-A to section B-B in the insets in FIGS. 29a and 29b, respectively. Depressing the trigger further pushes disk 310 (B) towards the now stationary disk 314 (C) and causes the tube to bend, as shown by comparing FIG. 28b to FIG. 28c. This action pushes the blade out of line with the endoscope axis and into contact with the tissue of interest. In the embodiment shown in FIGS. 29a-29c, the electrosurgical generator can energize the electrode when required, such as after the electrode has been positioned against the tissue to be cut. Applicants recognize that as the size of endoscopes has decreased, the availability of tools that operate in the smaller working channel of these endoscopes has been limited especially with respect to exhibiting sufficient structural integrity to cut and/or manipulate tougher tissues.

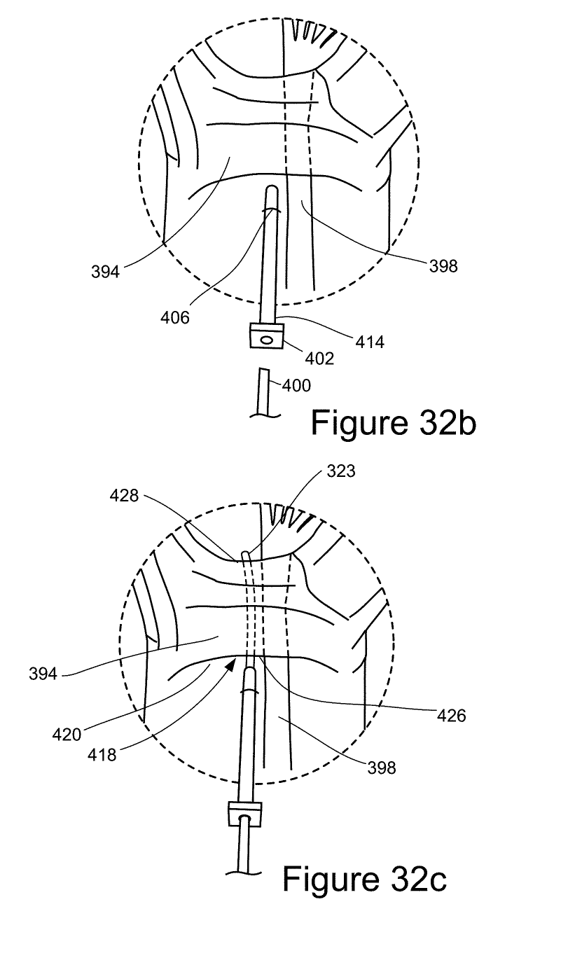

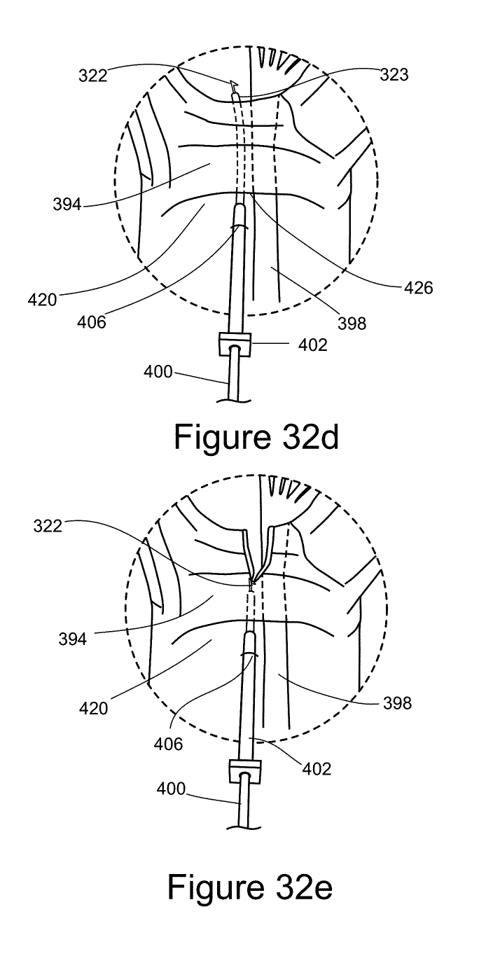

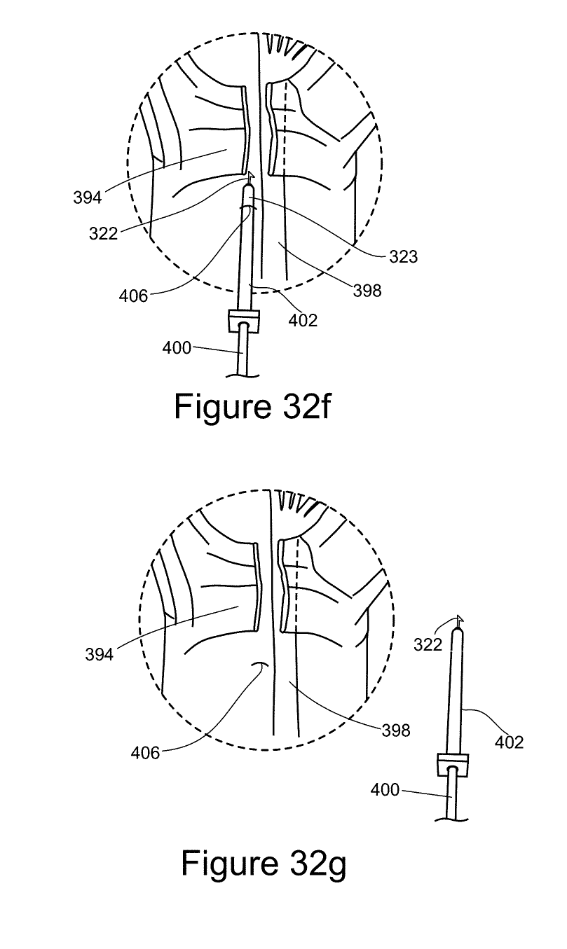

Referring now to FIG. 30 in conjunction with FIGS. 31a-31f, and 32a-32g, an embodiment of a method 350 is disclosed for correcting tissue sheath interference disorder in an anatomical joint. Method 350 can utilize endoscope 300 shown and described in FIGS. 28a-28c having the cutting blade knife; and 29a-29c having the electrosurgical electrode. Method 350 is discussed with respect to a finger joint 382 shown in FIGS. 31a-31f by way of non-limiting example. The tissue sheath interference disorder can be flexor tendinitis which is a condition in which a tendon 384 of a finger 386 becomes swollen or enlarged and catches on a tissue sheath 388, also referred to as a pulley, through which the tendon slides during movement of the finger. This can occur at the first pulley where the finger meets the hand as shown in FIGS. 31a-31c; and the procedure to correct this condition can be referred to as a “Trigger Finger Release” procedure. Method 350 is also discussed with respect to a wrist joint 392, shown in FIGS. 32a-32g, in which case the tissue sheath interference disorder can be carpal tunnel syndrome where a transverse carpal ligament 394 across the wrist on a palmar side of a hand 396 compresses or irritates one or more anatomical structures underneath the carpal ligament, such as tendons or median nerve 398.

Method 350 starts at 352 and proceeds to 354 where a hypodermic needle 400 and catheter 402 are inserted into tissue near the joint, see FIGS. 31a and 32a. The hypodermic needle can be fairly large gauge, such as 17-gauge and can have a distal end 404 having a cutting edge for puncturing the tissue creating a puncture 406. The needle and catheter are arranged such that the needle fits in a lumen 408 of the catheter (FIGS. 31b and 32b) and extends from a distal end 410 of the catheter at least to the extent to which the cutting edge of the needle can puncture the tissue. As the needle is inserted into the tissue, and needle guides the catheter along with the needle to a position near tissue sheath 388 or 394, FIGS. 31c and 32c respectively. The needle can be angled when inserted such that the lumen at end of the catheter is aimed between the sheath and the anatomical structure that the sheath restricts.

Method 350 then proceeds to 356 where the hypodermic needle is removed from the tissue and the catheter while the catheter is maintained in the tissue with the distal end of the catheter near the tissue sheath, as shown in FIGS. 31b and 32b. The hypodermic needle can be removed by pulling a proximal end 412 of the needle while holding a proximal end 414 of the catheter. Removing the needle from the catheter leaves the catheter lumen open.

Method 350 then proceeds to 358 where a distal end of a probe of an endoscope is inserted into the lumen at proximal end 414 of the catheter. The endoscope can be endoscope 300 and the probe can be probe 302 having distal end 323, shown in FIGS. 28a-28c and 29a-29c. As the probe is inserted, the catheter lumen guides the distal end of the probe to a gap 416 between tendon 384 and tissue sheath 388 (FIG. 31c) or a gap 418 between carpal ligament 394 and anatomical structure 420 that is under the carpal ligament (FIG. 32c). The insertion of the probe and/or distal end of the catheter can create or enlarge the gap.

Method 350 then proceeds to 360 where the tissue sheath is imaged with the probe to determine the position of the probe relative to the tissue sheath. The probe can be configured with a distal end similar to those shown in FIGS. 5 and 6 for imaging. The imaging can be continuous starting, for instance, when the probe is first inserted into the catheter and can continue until the probe is removed from the catheter when the procedure is complete. In addition to imaging the tissue sheath, other anatomical structures can be imaged including tendons, nerves, blood vessels, bones and other tissue that may require manipulation or be avoided. Imaging can be used to confirm that the distal end of the probe and the cutting tool are positioned properly before and/or after the cutting tool is extended from the probe. In another embodiment, imaging can be accomplished using ultrasound.

Method 350 then proceeds to 362 where the probe is moved longitudinally in the catheter lumen to extend the distal end of the probe from the distal end of the catheter and to interpose the probe under the tissue sheath. The probe can be extended until the distal end of the probe has moved from one end of the tissue sheath to the other end of the tissue sheath under the tissue sheath. For instance, the probe can be extended underneath tissue sheath 388, between the tissue sheath and tendon 384, from a first side 422 to a second side 424, as shown in FIG. 31c; and the probe can be extended underneath tissue sheath 394, between the tissue sheath and anatomical structure 420, from a first side 426 to a second side 428, as shown in FIG. 32c. While the probe is extended the distal end of the probe can be bent and/or rotated to direct the probe to the desired location.

Method 350 then proceeds to 364 where the cutting tool is extended from the distal end of the probe to the tissue sheath, as shown in FIGS. 31d and 32d. The cutting tool can be a knife blade, such as blade 322 shown in FIGS. 28a-28c, or can be an electrosurgical electrode, such as electrode 324 shown in FIGS. 29a-29c. The distal end of the probe can be biased against the tissue sheath by bending the end of the probe, as shown in FIGS. 28c and 29c, and such bias can be used to maintain the cutting tool against the tissue sheath during cutting. Biasing the end of the probe and therefore the cutting tool against the tissue sheath can achieve more efficient cutting. Since the cutting tool is actively pushing against the tissue sheath, it is less likely to move away from the sheath while cutting. The cutting tool can be extended from the end of the probe before, after or during the bending of the distal end of the probe. As shown in FIGS. 28a-28c, and 29a-29c, the endoscope can extend the cutting tool from the distal end and bend the distal end simultaneously which can be used to advantageously move the cutting tool to the tissue sheath and bias the cutting tool against the tissue sheath.

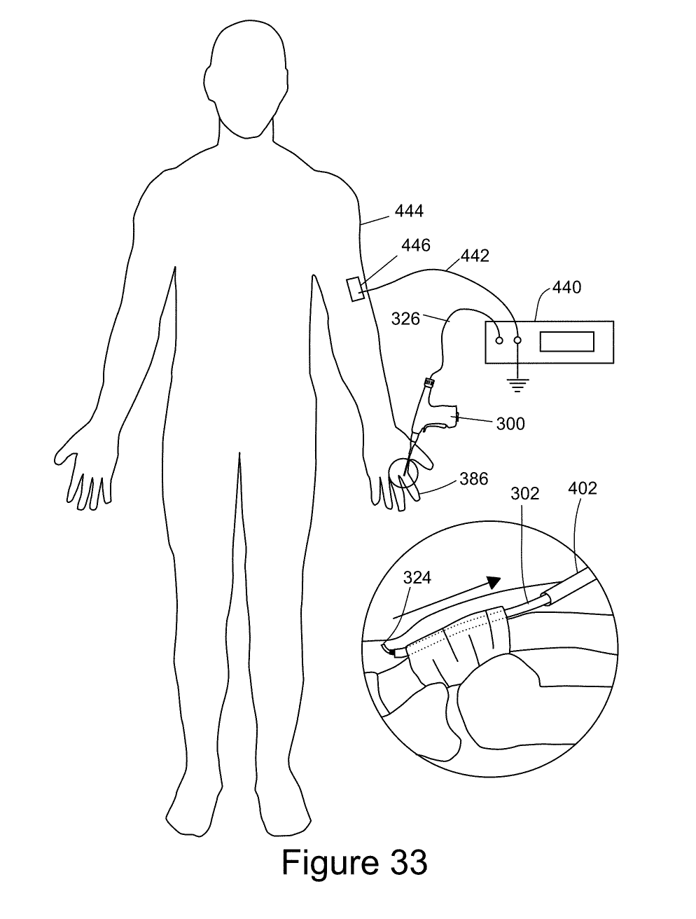

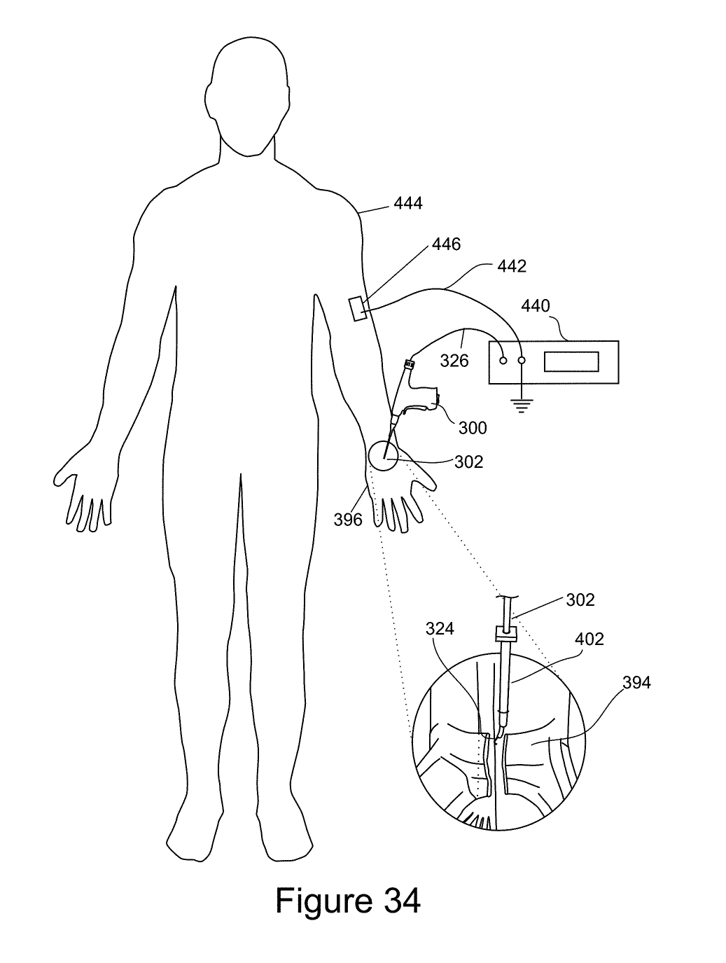

Method 350 then proceeds to 366 where the probe is pulled to move the distal end of the probe and the cutting tool toward the distal end of the catheter such that the cutting tool cuts the tissue sheath, as shown in FIGS. 31e, 32e and 32f. In the embodiment where the cutting tool is an electrode, such as specifically shown in FIGS. 33-38, the electrode can be energized with an electrosurgical generator 440 that includes a ground lead 442 that can attach to a patient 444 with a round patch 446. The electrode can be energized whenever appropriate, such as when the cutting tool is in contact with the tissue sheath and just before and during movement of the cutting tool towards the distal end of the catheter. A benefit of employing electrosurgery over a physical blade device is that the electrical current, power and waveform provided to the electrode from the electrosurgical generator can be altered to adjust for variations in the size and thickness of the tissue sheath. For instance, for cutting tissue a low-voltage, alternating current of hundreds of kilohertz to several megahertz is typically employed. Also, the current can be adjusted upward until cutting is achieved. Slightly depressing the trigger of endoscope 300 in FIGS. 29a-29c exposes electrode 324 while a full depression of the trigger bends the electrode out of line with the endoscope and, in this case, into contact with the tissue sheath.

The tissue sheath can be imaged before, during and after cutting to determine whether the tissue sheath was completely severed or if repeated passes with the cutting tool need to be made to completely sever the tissue sheath. Imaging can also be used to insure that other anatomical structures, such as tendon 384 and median nerve 398 are not damaged during the procedure. The method can continue once it is determined that the tissue sheath is completely severed.

Method 350 then proceeds to 368 where the probe and the catheter are removed from the tissue, as shown in FIGS. 31f and 32g. Prior to removing the probe and catheter, the cutting tool can be retracted and/or the electrode can be de-energized and the distal end of the probe can be straightened. The probe and the catheter can be removed together or the probe can be removed from the catheter and then the catheter can be removed from the tissue. Following 368, method 350 proceeds to 370 where the method ends.

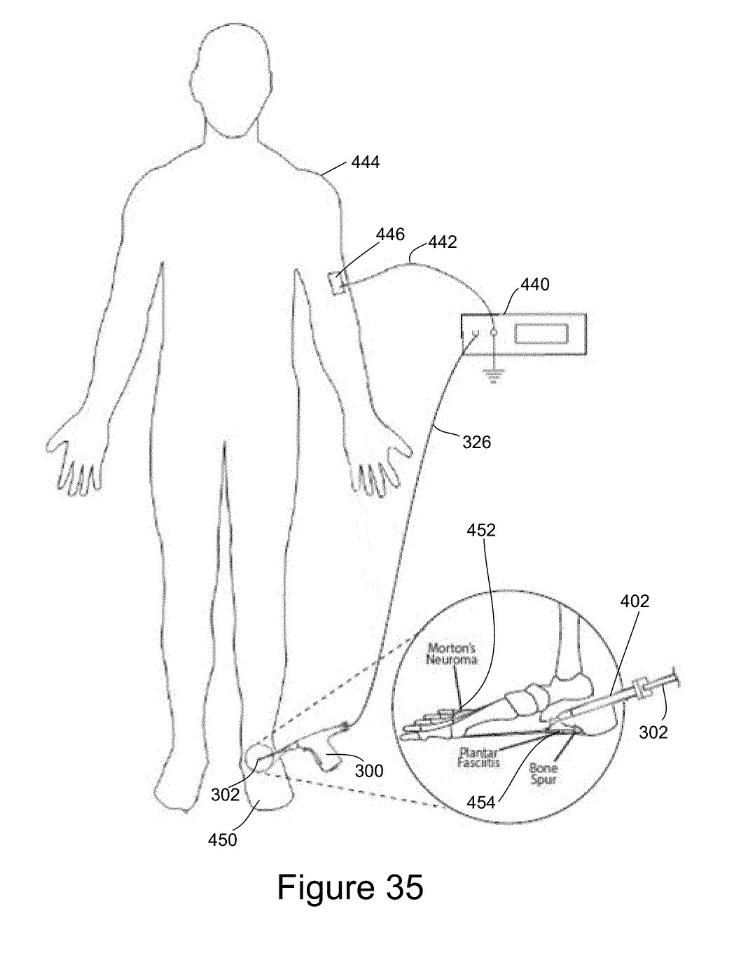

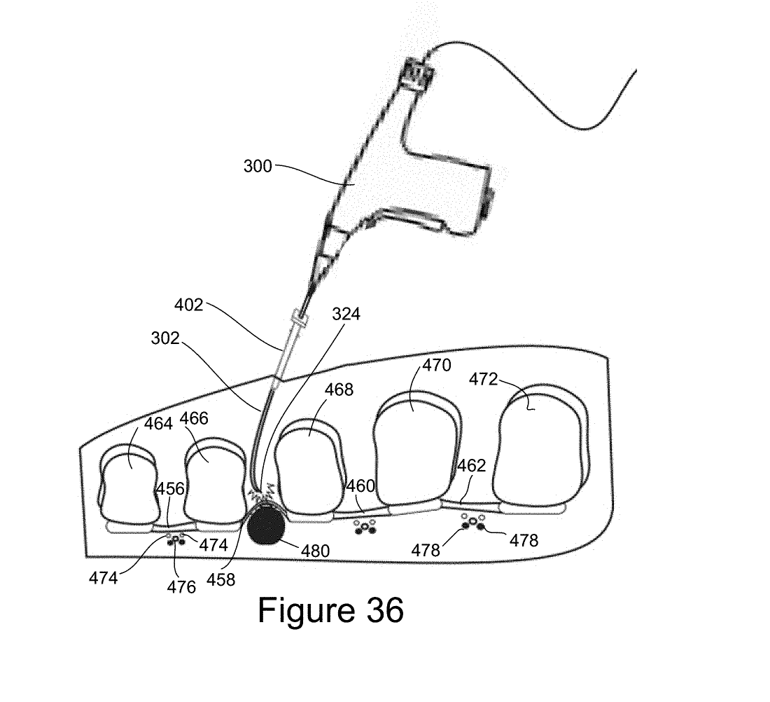

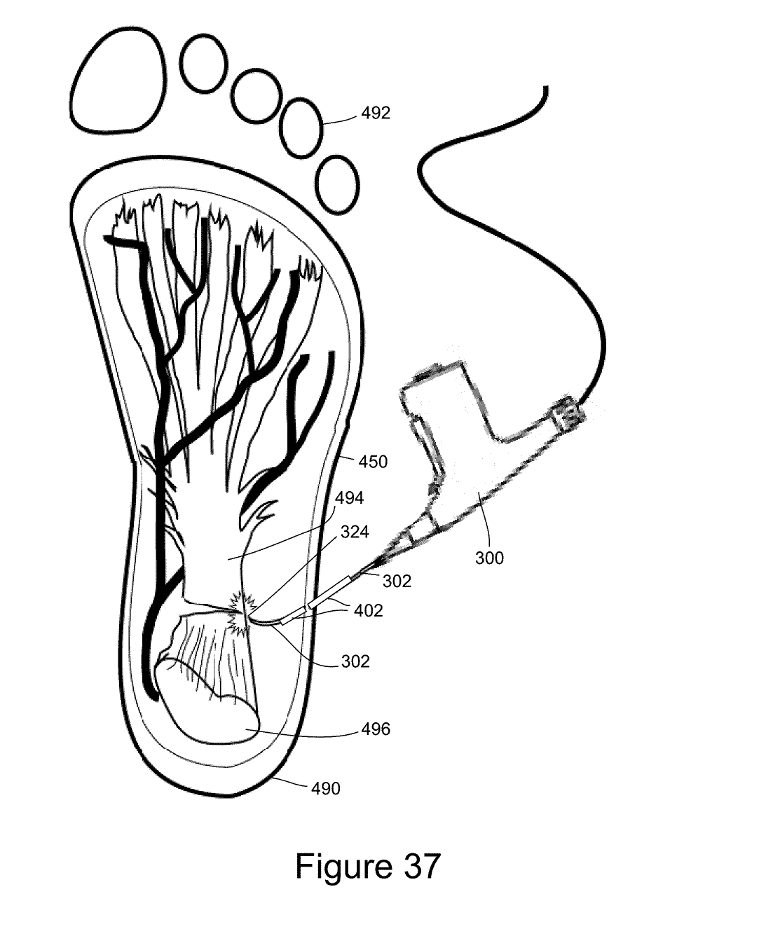

Although method 350 is discussed with respect to a finger joint 382 (FIGS. 31a to 31f) and a wrist joint 384 (FIGS. 32a to 32g), method 350 can be adapted for use for correcting tissue sheath interference disorders in other anatomical joints as well. For instance, as shown in FIGS. 35, 36 and 37, tissue sheath interference disorders can occur in the foot as well as the hand and wrist. FIG. 35 generically shows method 350 applied to the correction of a tissue sheath interference disorder in a joint of a foot 450 of patient 444. The disorder can be a Morton's neuroma (FIG. 36) which can occur towards the front portion 452 of the foot or plantar fasciitis which can occur towards the back portion 454 of the foot, FIG. 37.

Referring now to FIG. 36, a diagrammatic cross-section of foot 450 towards the front portion of the foot along is shown with endoscope 300. Foot 450 includes deep transverse metacarpal ligaments (DTML) 456, 458, 460 and 462 that extend between bones 464, 466, 468, 470, and 472. Below each DTML is a bundle of anatomical structures that includes two veins 474, one artery 476, and two nerves 478. As is typical in Morton's neuroma a tumorous growth 480 of one of the nerves between bones 466 and 468 illustrates how the size of these drastic tumorous growths can compress and irritate the surrounding structures. Using the technique described in method 350 electrode 324 can be introduced through catheter 402 to DTML 458 to electrosurgically sever the ligament and release the pressure on the veins, artery, and other nerve below DTML 458. A conventional Morton's neuroma release procedure involves a fully invasive open surgery, however, using the techniques described herein the Morton's neuroma release procedure can be accomplished without incision.

In an embodiment, a Morton's neuroma release procedure can involve prepping the patient's foot with Betadine cleansing solution, sterile draping, and an ankle tourniquet. Adhering an electrocautery grounding pad to the patient's lateral thigh. Injecting lidocaine 2 cm proximal to the inner digit webspace of the suspected Morton's neuroma on the dorsal aspect of the foot. Once anesthetized, introduce a sheathed 6Fr needle into the dorsum of foot at a 60° angle aiming distally. Under ultrasound guidance, position the needle tip at the approximate location of the deep transverse metatarsal ligament just above the neurovascular bundle and location of the neuroma. Remove the needle from the jacketed catheter lumen. Inject 2 mL of sterile saline solution through the catheter sheath for debris clearing and micro-insufflation. Insert the endoscope probe into the proximal end of the 6Fr catheter sheath and through to the distal end destination. Identify the deep transverse metatarsal ligament under direct visualization through the probe. Deploy the electrocautery electrode through the probe to the DTML and cut the DTML under visualization. After the ligament is completely incised, remove the probe from the catheter lumen. Additional sterile saline may be injected and suctioned for irrigation. Remove the catheter from the foot puncture site. Remove the tourniquet, assess the skin for any bleeding and apply small dressing to puncture site.

Referring now to FIG. 37, a diagrammatic cross-section of the base of foot 450 having plantar fasciitis is shown with endoscope 300. Foot 450 includes a heel 490 and toes 492, and a plantar fascia 494 attached to a calcaneus bone 496. Using the technique described in method 350, electrode 324 can be introduced through catheter 402 to the plantar fascia to electrosurgically sever the fascia.

In an embodiment, a plantar fasciitis procedure can involve prepping the patient's foot with Betadine cleansing solution, sterile draping, and an ankle tourniquet. Adhering an electrocautery grounding pad to the patient's lateral thigh. Injecting lidocaine at the plantar aspect of the heel and 2 cm proximal to the hind tip of the calcaneus on the medial aspect of the foot. Once anesthetized, introduce a sheathed 6Fr needle into the medial aspect of the foot near the calcaneal attachment of the plantar fascia aiming laterally. Under ultrasound guidance, position the needle tip below plantar fascia (between the fascia and the fat pad) near the calcaneal. Remove the needle from the jacketed catheter lumen. Inject 2 mL of sterile saline solution through the catheter sheath for debris clearing and micro-insufflation. Insert the endoscope probe into the proximal end of the 6Fr catheter sheath and through to the distal end destination. Identify the plantar fascia under direct visualization through the probe. Deploy the electrocautery electrode through the probe to the plantar fascia and cut the plantar fascia under visualization either completely or just the medial aspect to release nerve impingement. After the ligament is adequately incised, remove the probe from the catheter lumen. Additional sterile saline may be injected and suctioned for irrigation. Remove the catheter from the foot puncture site. Remove the tourniquet, assess the skin for any bleeding and apply small dressing to puncture site.

Current conventional arthroscopy techniques utilize relatively large rigid probes that are inserted into the joint through an incision. The site is insufflated, typically using sterile saline, to create an area around the joint so that the distal end of the rigid probe can be moved around to view the structure of the joint. Movement of the rigid probe through the incision as well as insufflation can cause unnecessary tissue damage which can increase healing time and can increase the risk of infection.

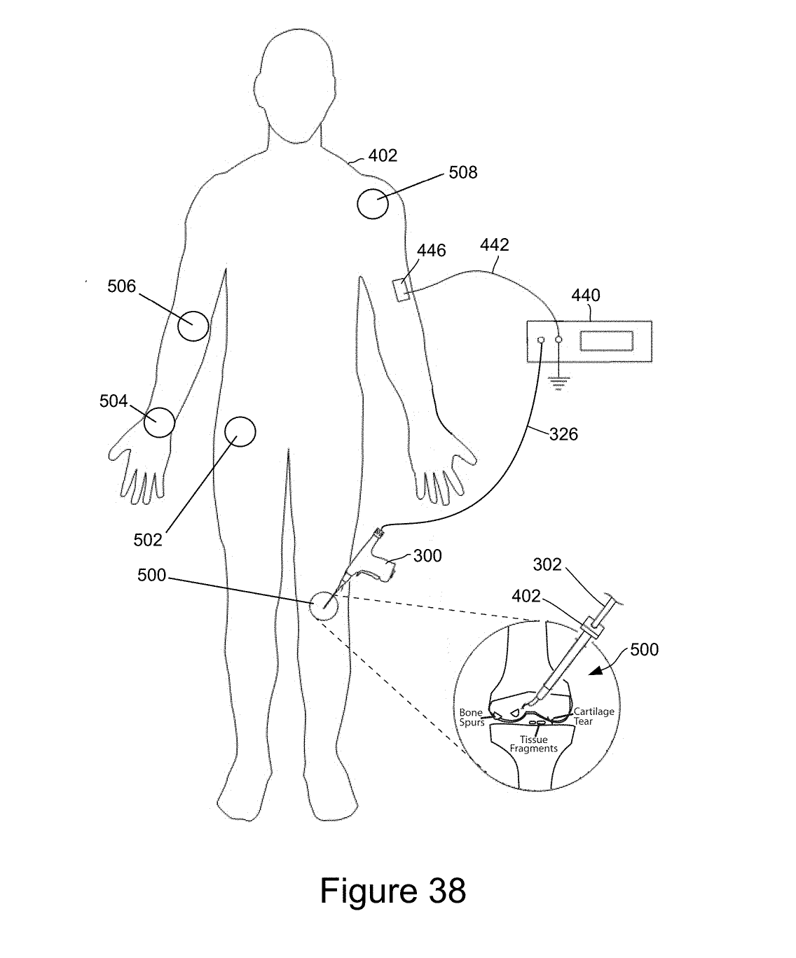

Referring now to FIG. 38, endoscopes having the functional aspects described herein can also be used beneficially for large joint arthroscopy and intervention. Because of the small size and the rotation and steering functionality, the endoscopes described herein can be used for visualization and intervention in large joints without the need for incisions, insufflation, or dilation. The large joints can include a knee joint 500, hip joint 502, wrist joint 504, elbow joint 506, and shoulder joint 508. The distal end of the endoscope probe can be inserted into the large joint using a needle and catheter and the distal end can be rotated and steered to visualize and/or treat different anatomical structures in the joint without having to insufflate or dilate to make room for the probe. For example, a probe having a cutting head can be inserted into the knee joint to excise or shave off frayed tissue, such as meniscus tissue. When electrocautery is used small pieces can be vaporized with the electrocautery so that they do not have to be removed from the site after they are cut off. These procedures can be visualized using the same probe and the same insertion catheter. The joints can be imaged using the catheter to view damage in the joint such as to determine whether or not a knee ligament, such as the ACL, is torn. These procedures can be performed in a doctor's office under a local anesthetic rather than having to undergo an MRI or other expensive imaging procedure.

In an embodiment, the knee arthroscopy procedure can involve prepping the patient's knee with Betadine cleansing solution, sterile draping, and a tourniquet above the knee. Placing the knee in a flexed position. Injecting lidocaine at the medial or lateral aspect of the knee into the skin and fat pad between the patella and tibia. Once anesthetized, introducing a sheath 6Fr needle, for smaller for a diagnostic probe only, into the medial or lateral aspect of the knee between the infra-patellar ligament and the patellar retinaculum aiming toward the center of the joint at a shallow angle. Once within the joint, remove the needle from the jacketed catheter lumen. Through the catheter lumen inject 2-4 cc of sterile saline solution for debris clearing and/or micro-insufflation. Insert endoscope probe into proximal end of catheter lumen and through to the distal end destination. Visualize the joint space for assessing any pathology such as damage to joint surface, torn ligaments, or torn meniscus. After completion of diagnostics, remove the probe and catheter from the skin puncture site. Prior to removal of the catheter, syringe suctioned may be applied to the end of the catheter lumen for removal of micro-insufflation saline. Remove the tourniquet, assess the skin for any bleeding and apply a small dressing to the puncture site.

In another embodiment, the knee arthroscopic procedure can involve deploying and electrocautery element through the endoscope probe for cauterization or “shaving” of small tissue frays or bone spurs under visualization. The electrocautery grounding pad can be adhered to the patient's lateral thigh. Sterile saline may be injected and suctioned for irrigation. A biopsy or grasping tool may be deployed through the endoscope probe for tissue sampling or tissue removal. The catheter may be positioned at a precise location needed for injection of bone stem cells, chondrocytes, platelet rich plasma, and the like. The endoscope probe can be removed from the catheter lumen and the biomaterial can be injected through the lumen. Once the intervention has been completed, the endoscope probe and the catheter can be removed.

Various embodiments of endoscopes are disclosed which incorporate several features including a rotation and steering mechanism. An actuator controller is disclosed that significantly improves the tactile response of the endoscope to steering and tool engagement, particularly that the effects of steering and rotation do not impact the characteristics of tool engagement. An endoscope probe sheath is disclosed with a reniform shaped (i.e. kidney shaped) working channel which maximizes the cross sectional area of the working channel while minimizing the cross-sectional height; and a micro tool design which maximizes the utility of the reniform shaped working channel. Also described is an endoscope which incorporates a physical device for cutting tissue or any other material via an edge which is integral to an endoscopic steering mechanism. Further described is an endoscope which incorporates an electrical device to cut, coagulate, desiccate or fulgurate tissue via an electrode, and which is integral to an endoscope steering mechanism that can be controlled by the operator.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. An endoscope comprising:
a probe including an imaging fiber bundle for transferring a light image, the imaging fiber bundle having a distal end for receiving the light image and a proximal end portion extending out of the probe for emitting the light image; and
a handle arrangement connected to the probe and configured to support part of the proximal end portion of the imaging fiber bundle for twisting therealong responsive to rotating the probe including the distal end of the imaging fiber bundle relative to the handle arrangement.

2. The endoscope as defined in claim 1, wherein the handle arrangement is configured to selectively twist the imaging fiber bundle by at least 177° in a first rotational direction and in a second, opposite rotational direction.

3. The endoscope as defined in claim 1, wherein the handle arrangement includes a cavity which receives the part of the proximal end portion of the imaging fiber bundle for said twisting.

4. The endoscope as defined in claim 3, wherein the imaging fiber bundle enters the cavity from a Y-extension of the handle and passes through said cavity to enter the probe.

5. The endoscope as defined in claim 1, wherein the imaging fiber bundle is supported at opposite ends of the proximal end portion such that one end of the imaging fiber bundle co-rotates with the probe and the other end is non-rotationally fixed to the handle.

6. The endoscope as defined in claim 1, wherein the handle arrangement includes a forward end that supports a manipulation cone that co-rotates with the probe and is arranged for manual rotation of the probe and the fiber bundle.

7. The endoscope as defined in claim 1, wherein the handle arrangement includes a rear end that supports a knob that co-rotates with the probe and is arranged for manual rotation of the probe and fiber bundle.

8. The endoscope as defined in claim 7, wherein the handle arrangement defines a central axis along which a working channel is defined and which extends through the knob to the probe.

9. The endoscope as defined in claim 8, wherein the handle arrangement includes a cavity which receives the part of the proximal end portion of the imaging fiber bundle for said twisting around the working channel.

10. An endoscope, comprising:
a probe configured for insertion into tissue;
an imaging fiber bundle supported by the probe and having a distal end, a proximal end, and a length therebetween, the imaging fiber bundle configured for receiving a light image using the distal end, transferring the light image from the distal end to the proximal end, and emitting the light image from the proximal end; and
a handle arrangement connected to the probe and the imaging fiber bundle, the handle arrangement configured for co-rotating the probe and a distal portion of the imaging fiber bundle while holding a proximal portion of the imaging fiber bundle substantially without rotation to rotate the light image along said length responsive to the probe rotation such that the light image as emitted from the proximal end of the imaging fiber bundle is rotated relative to the light image received at the distal end of the imaging fiber bundle.

11. The endoscope as defined in claim 10, wherein the handle arrangement is configured for co-rotating the probe and distal end of the imaging fiber bundle relative to the proximal portion of the imaging fiber bundle.

12. The endoscope as defined in claim 11, wherein the probe defines a working channel and the distal end of the imaging fiber bundle is maintained in a fixed orientation relative to the working channel such that the light image at the proximal end of the imaging fiber bundle is provided from a viewpoint that is fixed with respect to the working channel.

13. An endoscope, comprising:
a probe including a distal end configured for insertion into tissue and a proximal end configured for use outside of the tissue, the probe including a substantially circular exterior cross-sectional shape perpendicular to a center axis which extends between the proximal and distal ends of the probe, the probe including an imaging fiber bundle having a substantially circular cross-sectional shape that is sized and positioned within the probe such that the center axis of the probe is within the imaging fiber bundle, and the probe defines a working channel, spaced apart from the imaging fiber bundle, the working channel including a reniform cross-sectional shape for receiving at least one of a plurality of endoscopic tools having a complementary reniform exterior cross section and for guiding a received one of the endoscopic tools from the proximal end of the probe to the distal end of the probe.

14. The endoscope defined by claim 13 wherein the reniform cross-sectional shape maintains the endoscopic tool in a fixed rotational orientation relative to the probe.

15. The endoscope defined by claim 13 wherein the received tool includes at least two components that are held in operative communication by the reniform cross-sectional shape.

16. The endoscope defined by claim 13 wherein the received tool is a biopsy tool.

17. An endoscope, comprising:
a probe including a distal end configured for insertion into tissue and a proximal end configured for use outside of the tissue, the probe defining a working channel for guiding endoscopic tools from the proximal end of the probe to the distal end of the probe; and
an endoscope tool configured for insertion through the working channel to a surgical site in the tissue and for tool actuation to manipulate tissue at the surgical site, the tool and the working channel including complementary configurations which cooperate for the tool actuation of the endoscope tool.

18. The endoscope of claim 17 wherein the working channel is reniform in cross-sectional shape.

19. The endoscope defined by claim 17 wherein the received tool includes at least two components that are held in operative communication by the reniform cross-sectional shape.

20. An endoscope, comprising:
a tool assembly having a tool head that is configured for selective movement to manipulate tissue and a tool head actuator that is connected to selectively move the tool head using a cable assembly having a cable sheath and an inner cable that moves longitudinally in the cable sheath;
an elongated probe including a distal end configured for insertion into tissue and a proximal end configured for use outside of the tissue, the probe defining a working channel for guiding the tool head from the proximal end of the probe to the distal end of the probe while the tool head actuator remains outside of the tissue; and
a handle assembly connected to the probe, the handle assembly including a handle body, a trigger arrangement and a latching mechanism, the latching mechanism configured for selectively connecting the tool assembly to the handle assembly and the trigger arrangement is configured for an actuating movement relative to the handle body to actuate the cable assembly to bend the probe near the distal end of the probe and an unlatching movement relative to the handle body to control the latching mechanism to disconnect the tool assembly from the handle assembly.

21. The endoscope as defined in claim 20, wherein the actuation of the cable assembly to bend the probe operates independently from the tool head actuator such that bending the probe does not change the operational status of the tool head.

22. An endoscope, comprising:
a tool assembly having a tool head that is configured for selective movement to manipulate tissue and a tool head actuator that is connected to selectively move the tool head using a cable assembly having a cable sheath and an inner cable that moves longitudinally in the cable sheath;
an elongated probe including a distal end configured for insertion into tissue and a proximal end configured for use outside of the tissue, the probe defining a working channel for guiding the tool head from the proximal end of the probe to the distal end of the probe while the tool head actuator remains outside of the tissue and the distal end is configured for selective bending; and
a handle assembly operatively coupled to the probe, the handle assembly including a handle body and a trigger arrangement that is configured for an actuating movement relative to the handle body to actuate the cable assembly to initially extend the tool head from the probe and, thereafter, bend the distal end of the probe.

23. The endoscope as defined by claim 22 wherein the tool head comprises a cutting blade for cutting tissue proximate to the bent probe.

24. The endoscope as defined by claim 23 wherein the handle is configured for manipulation to rotate the probe relative to the handle probe and thereby rotate the distal end of the probe.
